SEQUENCE LISTING (50 sequences)

SEQ ID NO: 1 - protein, 5 aa, synthetic construct
  REGION 1..5: note = 28C8 HCDR1
SYAMN

SEQ ID NO: 2 - protein, 16 aa, synthetic construct
  REGION 1..16: note = 28C8 HCDR2
IIYDSGSTYYANWAKG

SEQ ID NO: 3 - protein, 5 aa, synthetic construct
  REGION 1..5: note = 28C8 HCDR3
GYLDI

SEQ ID NO: 4 - protein, 13 aa, synthetic construct
  REGION 1..13: note = 28C8 LCDR1
QSSKSVYNNKWLS

SEQ ID NO: 5 - protein, 7 aa, synthetic construct
  REGION 1..7: note = 28C8 LCDR2
GASTLAS

SEQ ID NO: 6 - protein, 11 aa, synthetic construct
  REGION 1..11: note = 28C8 LCDR3
AGGYSISSDNA

SEQ ID NO: 7 - protein, 129 aa, synthetic construct
  REGION 1..129: note = 28C8 VH AA
METGLRWLLLVAVLKGVQCQSVEESGGRLVTPGTPLTLTCTVSGFSLSSYAMNWVRQAPGKGLEYIGIIYDSGSTYYANWAKGRFTISKTSTTVDLKITSPTTEDTATYFCARGYLDIWGPGTLVTVSS

SEQ ID NO: 8 - protein, 133 aa, synthetic construct
  REGION 1..133: note = 28C8 VL AA
MDTRAPTQLLGLLLLWLPGARCAAVLTQTPSPVSAAVGGTVSISCQSSKSVYNNKWLSWLQQKPGQPPKLLIYGASTLASGVPSRFKGSGSGTEFTLTISDVQCDDAATYYCAGGYSISSDNAFGGGTEVVVK

SEQ ID NO: 9 - DNA, 387 bp, synthetic construct
  misc_feature 1..387: note = 28C8 VH DNA
  source 1..387: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcagtagctatgcaatgaactgggtccgccaggctccaggaaaggggctggaatacatcggaattatttatgatagtggtagcacatactacgcgaactgggcgaaaggccgattcaccatctccaaaacctcgaccacggtggatctgaaaatcaccagtccgacaaccgaggacacggccacctatttctgtgccagaggttatttagacatctggggcccaggcaccctggtcaccgtctcctca

SEQ ID NO: 10 - DNA, 399 bp, synthetic construct
  misc_feature 1..399: note = 28C8 VL DNA
  source 1..399: mol_type = other DNA
atggacacgagggcccccactcaactgctggggctcctgctgctctggctcccaggtgccagatgtgccgccgtgctgacccagactccatctcccgtgtctgcagctgtgggaggcacagtcagcatcagttgccagtccagtaagagtgtttataataacaagtggctatcctggcttcagcagaaaccagggcagcctcccaagctcctgatctatggtgcatccactctggcatctggggtcccatcgcggttcaaaggcagtggatctgggacagagttcactctcaccatcagcgacgtgcagtgtgacgatgctgccacttactactgtgcgggcggttatagtattagtagtgataatgctttcggcggagggaccgaggtggtggtcaaa

SEQ ID NO: 11 - protein, 5 aa, synthetic construct
  REGION 1..5: note = 29D2 HCDR1
SYAMN

SEQ ID NO: 12 - protein, 16 aa, synthetic construct
  REGION 1..16: note = 29D2 HCDR2
IIYDSGTTYYASWAKG

SEQ ID NO: 13 - protein, 5 aa, synthetic construct
  REGION 1..5: note = 29D2 HCDR3
AALDI

SEQ ID NO: 14 - protein, 13 aa, synthetic construct
  REGION 1..13: note = 29D2 LCDR1
QASKSVYNNNWLS

SEQ ID NO: 15 - protein, 7 aa, synthetic construct
  REGION 1..7: note = 29D2 LCDR2
GASTLAS

SEQ ID NO: 16 - protein, 11 aa, synthetic construct
  REGION 1..11: note = 29D2 LCDR3
AGGYGSSSDNA

SEQ ID NO: 17 - protein, 129 aa, synthetic construct
  REGION 1..129: note = 29D2 VH AA
METGLRWLLLVAVLKGVQCQSVEESGGRLVTPGTPLTLTCTASGFSLSSYAMNWVRQAPGKGLEYIGIIYDSGTTYYASWAKGRFTISKTSTTVDLKITSPTTEDTATYFCARAALDIWGPGTLVTVSS

SEQ ID NO: 18 - protein, 133 aa, synthetic construct
  REGION 1..133: note = 29D2 VL AA
MDTRAPTQLLGLLLLWLPGATFAAVLTQTPSPVSAAVGGTVSISCQASKSVYNNNWLSWVQQTPGQPPKLLIYGASTLASGVPSRFKGSGSGTQFTLTISDVQCDDAATYYCAGGYGSSSDNAFGGGTEVVVE

SEQ ID NO: 19 - DNA, 387 bp, synthetic construct
  misc_feature 1..387: note = 29D2 VH DNA
  source 1..387: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagcctctggattctccctcagtagctatgcaatgaactgggtccgccaggctccagggaaggggctggaatacatcggaatcatttatgatagtggcaccacatactacgcgagctgggcgaaaggccgattcaccatctccaaaacctcgaccacggtggatctgaaaatcaccagtccgacaaccgaagacacggccacttatttctgtgccagagcagcattggacatctggggcccaggcaccctggtcaccgtctcctca

SEQ ID NO: 20 - DNA, 399 bp, synthetic construct
  misc_feature 1..399: note = 29D2 VL DNA
  source 1..399: mol_type = other DNA
atggacacgagggcccccactcagctgctggggctcctgctgctctggctcccaggtgccacatttgccgccgtgctgacccagactccgtctcccgtgtctgcagctgtgggaggcacagtcagcatcagttgccaggccagtaagagtgtttataataacaactggttatcctgggttcagcagacaccagggcagcctcccaagctcctgatctatggtgcatccactctggcatctggggtcccatcgcggttcaaaggcagtggatctgggacacagttcactctcaccattagcgacgtgcagtgtgacgatgctgccacttactactgtgcaggcggttatggtagtagtagtgataatgctttcggcggagggaccgaggtggtggtcgaa

SEQ ID NO: 21 - protein, 5 aa, synthetic construct
  REGION 1..5: note = 35D6 HCDR1
SHWMS

SEQ ID NO: 22 - protein, 16 aa, synthetic construct
  REGION 1..16: note = 35D6 HCDR2
IISSSGSTDYVSWAKG

SEQ ID NO: 23 - protein, 8 aa, synthetic construct
  REGION 1..8: note = 35D6 HCDR3
DLGYSTNI

SEQ ID NO: 24 - protein, 11 aa, synthetic construct
  REGION 1..11: note = 35D6 LCDR1
QASQSIGSYLA

SEQ ID NO: 25 - protein, 7 aa, synthetic construct
  REGION 1..7: note = 35D6 LCDR2
DASTLAS

SEQ ID NO: 26 - protein, 12 aa, synthetic construct
  REGION 1..12: note = 35D6 LCDR3
QQGYSSSGVDNA

SEQ ID NO: 27 - protein, 132 aa, synthetic construct
  REGION 1..132: note = 35D6 VH AA
METGLRWLLLVAVLKGVQCQSVEESGGRLVTPGTPLTLTCTVSGFSLSSHWMSWVRQAPGKGLEWIGIISSSGSTDYVSWAKGRFTISKTSTTVDLKITSPTTEDTATYFCARDLGYSTNIWGPGTLVTVSL

SEQ ID NO: 28 - protein, 132 aa, synthetic construct
  REGION 1..132: note = 35D6 VL AA
MDTRAPTQLLGLLLLWLPGARCAYDMTQTPASVEVTVGGTVTIKCQASQSIGSYLAWYQQKPGQRPKLLIYDASTLASGVSSRFKGSGSGTQFTLTISGVECADAATYYCQQGYSSSGVDNAFGGGTEVVVK

SEQ ID NO: 29 - DNA, 396 bp, synthetic construct
  misc_feature 1..396: note = 35D6 VH DNA
  source 1..396: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggattctccctgagtagccattggatgagctgggtccgccaggctccagggaaggggctggaatggatcggaatcatcagtagtagtggtagtacggactacgtgagctgggcgaaaggccgattcaccatctccaaaacctcgaccacggtggatctgaaaatcaccagtccgacaaccgaggacacggccacctatttctgtgccagagatctgggttatagtactaacatctggggcccaggcaccctggtcaccgtctcctta

SEQ ID NO: 30 - DNA, 396 bp, synthetic construct
  misc_feature 1..396: note = 35D6 VL DNA
  source 1..396: mol_type = other DNA
atggacacgagggcccccactcagctgctggggctcctgctgctctggctcccaggtgccagatgtgcctatgatatgacccagactccagcctctgtggaggtaactgtgggaggcacagtcaccatcaagtgccaggccagtcagagcattggtagctacttagcctggtatcagcagaaaccaggacagcgtcccaagctcctgatctacgatgcatccactctggcatctggggtctcatcgcggttcaaaggcagtggatctgggacacagttcactctcaccatcagcggcgtggagtgtgccgatgctgccacttactactgtcaacagggttatagtagtagtggtgttgataatgctttcggcggagggaccgaggtggtggtcaaa

SEQ ID NO: 31 - protein, 5 aa, synthetic construct
  REGION 1..5: note = 76G4 HCDR1
SYYMF

SEQ ID NO: 32 - protein, 16 aa, synthetic construct
  REGION 1..16: note = 76G4 HCDR2
YIGDGGYTSYATWAKG

SEQ ID NO: 33 - protein, 8 aa, synthetic construct
  REGION 1..8: note = 76G4 HCDR3
DGFTSGSI

SEQ ID NO: 34 - protein, 13 aa, synthetic construct
  REGION 1..13: note = 76G4 LCDR1
QSSKNVYNNNWLS

SEQ ID NO: 35 - protein, 7 aa, synthetic construct
  REGION 1..7: note = 76G4 LCDR2
GTSTLAS

SEQ ID NO: 36 - protein, 11 aa, synthetic construct
  REGION 1..11: note = 76G4 LCDR3
AGGYSSSSDNG

SEQ ID NO: 37 - protein, 133 aa, synthetic construct
  REGION 1..133: note = 76G4 VH AA
METGLRWLLLVAVLKGVQCQSVEESGGRLVTPGTPLTLTCTASGFSLSSYYMFWVRQAPGKGLEYIGYIGDGGYTSYATWAKGRFTISKTSSTTVDLKITSPTTEDTATYFCARDGFTSGSIWGPGTLVTVSS

SEQ ID NO: 38 - protein, 133 aa, synthetic construct
  REGION 1..133: note = 76G4 VL AA
MDTRAPTQLLGLLLLWLPGATFAAVLTQTPSPVSTAVGGTVTISCQSSKNVYNNNWLSWFQQKPGQPPKLLIYGTSTLASGVPSRFKGSGSGTQFTLTISDVQCDDAATYYCAGGYSSSSDNGFGAGTEVVVK

SEQ ID NO: 39 - DNA, 399 bp, synthetic construct
  misc_feature 1..399: note = 76G4 VH DNA
  source 1..399: mol_type = other DNA
atggagactgggctgcgctggcttctcctggtcgctgtgctcaaaggtgtccagtgtcagtcggtggaggagtccgggggtcgcctggtcacgcctgggacacccctgacactcacctgcacagcctctggattctccctcagtagctactacatgttctgggtccgccaggctccagggaaggggctagaatacatcggatacattggtgatggtggttacacatcctacgcgacctgggcaaaaggccgattcaccatctccaaaacctcgtcgaccacggtggatctgaaaatcaccagtccgacaaccgaggacacggccacctatttctgtgccagagacggttttacttctggtagcatttggggcccaggcaccctggtcaccgtttcctca

SEQ ID NO: 40 - DNA, 399 bp, synthetic construct
  misc_feature 1..399: note = 76G4 VL DNA
  source 1..399: mol_type = other DNA
atggacacgagggcccccactcagctgctggggctcctgctgctctggctcccaggtgccacatttgccgccgtgctgacccagactccatctcccgtgtctacagctgtgggaggcacagtcaccatcagttgccagtccagtaagaatgtttataataacaactggttatcctggtttcagcagaaaccagggcagcctcccaagctcctcatctatggtacatccacgctggcgtctggggtcccatcgcggttcaaaggcagtggatctgggacacagttcactctcaccatcagcgacgtgcagtgtgacgatgctgccacttactactgtgcaggcggttatagtagtagtagtgataatggtttcggcgcagggaccgaggtggtggtcaaa

SEQ ID NO: 41 - protein, 5 aa, synthetic construct
  REGION 1..5: note = 77G10 HCDR1
SYYMS

SEQ ID NO: 42 - protein, 16 aa, synthetic construct
  REGION 1..16: note = 77G10 HCDR2
IISSSGSTDYASWAKG

SEQ ID NO: 43 - protein, 5 aa, synthetic construct
  REGION 1..5: note = 77G10 HCDR3
GWLDL

SEQ ID NO: 44 - protein, 13 aa, synthetic construct
  REGION 1..13: note = 77G10 LCDR1
QSSKSVYNNNWLS

SEQ ID NO: 45 - protein, 7 aa, synthetic construct
  REGION 1..7: note = 77G10 LCDR2
GASTLAS

SEQ ID NO: 46 - protein, 11 aa, synthetic construct
  REGION 1..11: note = 77G10 LCDR3
AGGYSSSTDNA

SEQ ID NO: 47 - protein, 129 aa, synthetic construct
  REGION 1..129: note = 77G10 VH AA
METGLRWLLLVAVLKGVQCQSLEESGGRLVTPGTPLTLTCTASGFSLSSYYMSWVRQAPGKGLEYIGIISSSGSTDYASWAKGRFTISKTSTTVDLKITSPTTEDTATYFCARGWLDLWGQGTLVTVSS

SEQ ID NO: 48 - protein, 133 aa, synthetic construct
  REGION 1..133: note = 77G10 VL AA
MDTRAPTQLLGLLLLWLPGATFAAVLTQTPSPVSAAVGGTVSISCQSSKSVYNNNWLSWLQQKPGQPPKLLIHGASTLASGVPSRFKGSGSGTQFTLTISDVQCDDAATYYCAGGYSSSTDNAFGGGTEVVVK

SEQ ID NO: 49 - DNA, 387 bp, synthetic construct
  misc_feature 1..387: note = 77G10 VH DNA
  source 1..387: mol_type = other DNA
atggagactgggctgcgctggcttctcctggtcgctgtgctcaaaggtgtccagtgtcagtcgctggaggagtccgggggtcgcctggtcacgcctgggacacccctgacactcacctgcacagcctctggattctccctcagtagttactacatgagctgggtccgccaggctccagggaaggggctggaatacatcggaatcattagtagtagtggttccacggactacgcgagctgggcgaaaggccgattcaccatctccaaaacctcgaccacggtggatctgaaaatcaccagtccgacaaccgaggacacggccacctatttctgtgccagaggctggttggatctctggggccagggcaccctggtcaccgtctcttca

SEQ ID NO: 50 - DNA, 399 bp, synthetic construct
  misc_feature 1..399: note = 77G10 VL DNA
  source 1..399: mol_type = other DNA
atggacacgagggcccccactcagctgctggggctcctgctgctctggctcccaggtgccacatttgccgccgtgctgacccagactccatctcccgtgtctgcagctgtgggaggcacagtcagcatcagttgccagtccagtaagagtgtttataataacaactggttatcctggcttcagcagaaaccagggcagcctcccaagctcctgatccatggtgcatccactctggcatctggggtcccatcgcggttcaaaggcagtggatctgggacacagttcactctcaccatcagcgacgtgcagtgtgacgatgctgccacttactactgtgcaggcggttatagtagtagtactgataatgctttcggcggagggaccgaggtggtggtcaaa

(19) United States
(12) Patent Application Publication
(10) Pub. No.: US 2025/0026859 A1
(43) Pub. Date: Jan. 23, 2025

(54) Anti-DXd Antibodies and Methods of Use

(71) Applicant: BeiGene, Ltd., Grand Cayman (KY)

(72) Inventors: Dan Li, Beijing (CN); Xiaoyan Tang, Beijing (CN); Charng-Sheng Tsai, Beijing (CN); Mei-Hsuan Tsai, Beijing (CN)

(73) Assignee: BeiGene, Ltd., Grand Cayman (KY)

(21) Appl. No.: 18/782,489

(22) Filed: Jul. 24, 2024

Related U.S. Application Data
(63) Continuation of application No. PCT/CN2023/073187, filed on Jan. 19, 2023.

(30) Foreign Application Priority Data
Jan. 26, 2022 (WO) PCT/CN2022/073949

(51) Int. Cl. C07K 16/44

(52) U.S. Cl. CPC C07K 16/44; C07K 2317/92

(57) ABSTRACT

The present disclosure provides antigen-binding fragments thereof that bind to DXd, and use of the antibody for detection and assay of DXd.

### CROSS-REFERENCE TO RELATED APPLICATIONS

This application is a continuation of International Application No. PCT/CN2023/073187, filed on Jan. 19, 2023, which claims priority to International Application No. PCT/CN2022/073949, filed on Jan. 26, 2022, the disclosures of each of which are hereby incorporated by reference in their entireties.

### FIELD OF THE DISCLOSURE

Disclosed herein are antibodies that specifically bind to topoisomerase inhibitors such as camptothecin and its derivatives such as exatecan (DXd) as well as isolated nucleic acids, vectors and host cells. These antibodies can be used to determine concentrations of topoisomerase inhibitors when these molecules are used in clinical settings, for example, when a topoisomerase inhibitor is conjugated to an antibody in an antibody drug conjugate (ADC) format.

### SEQUENCE LISTING

The instant application contains a Sequence Listing, which has been submitted electronically in XML format and is hereby incorporated by reference in its entirety. Said XML copy, created on Jan. 13, 2023, is named “BGB26501-01PCT sequence listings_ST26.xml” and is 67,801 bytes in size.

### BACKGROUND

Antibody drug conjugates (ADCs) are antibody molecules that are conjugated to a small molecule drug via a linker. The antibody portion of an ADC is specific for a particular antigen on a cell, provides specific binding to that antigen and allows delivery of the drug to the cell. The drug then has the desired effect on the cell, for example, cell death. In developing ADCs, the ability to determine the pharmacodynamics or pharmacokinetics (PD/PK) of the drug is of interest because the drug is being delivered via the ADC instead of the administration of the drug as a single agent at a known quantity (e.g., 10 mg/kg). The drug is often conjugated to the antibody in a drug to antibody ratio (DAR), wherein multiple drug moieties are conjugated to a single antibody. For example, a DAR of four indicates that there are four drug moieties per each antibody molecule.

One solution to determine PD/PK of the drug is to create an antibody that recognizes the drug. As antibodies are specific for their target, an antibody created against the drug would recognize this portion of the ADC and a separate antibody that recognizes the antibody portion of the ADC could be utilized to determine the concentration of the each in a sample after administration of the ADC. The detection of the anti-drug antibodies could be done by ELISA assay, immunohistochemistry or by another other means used to quantitate antibody-antigen complexes. Thus, there is need in the art for antibodies that determine concentration and/or PD/PK of a drug moiety which leads to data on administration of the ADC.

### SUMMARY OF THE DISCLOSURE

The present disclosure is directed to anti-DXd antibodies and antigen-binding antibody fragments thereof that specifically bind DXd.

The present disclosure encompasses the following embodiments.

An antibody or antigen binding antibody fragment thereof which specifically binds DXd.

1. An antibody or antigen-binding fragment thereof, comprising:
(i) a heavy chain variable region that comprises (a) a HCDR1 (Heavy Chain Complementarity Determining Region 1) of SEQ ID NO: 11, (b) a HCDR2 of SEQ ID NO: 12 and (c) a HCDR3 of SEQ ID NO: 13 and a light chain variable region that comprises: (d) a LCDR1 (Light Chain Complementarity Determining Region 1) of SEQ ID NO 14, (e) a LCDR2 of SEQ ID NO: 15, and (f) a LCDR3 of SEQ ID NO: 16;
(ii) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 4, (e) a LCDR2 of SEQ ID NO: 5, and (f) a LCDR3 of SEQ ID NO: 6;
(iii) a heavy chain variable region that comprises (a) a HCDR1 of SEQ ID NO: 21, (b) a HCDR2 of SEQ ID NO: 22 and (c) a HCDR3 of SEQ ID NO: 23; and a light chain variable region that comprises: (d) a LCDR1 of SEQ ID NO: 24, (e) a LCDR2 of SEQ ID NO:25, and (f) a LCDR3 of SEQ ID NO: 26;
(iv) a heavy chain variable region that comprises (a) a HCDR1 of SEQ ID NO: 31, (b) a HCDR2 of SEQ ID NO: 32 and (c) a HCDR3 of SEQ ID NO: 33; and a light chain variable region that comprises: (d) a LCDR1 of SEQ ID NO: 34, (e) a LCDR2 of SEQ ID NO:35, and (f) a LCDR3 of SEQ ID NO: 36; or
(v) a heavy chain variable region that comprises (a) a HCDR1 of SEQ ID NO: 41, (b) a HCDR2 of SEQ ID NO: 42 and (c) a HCDR3 of SEQ ID NO: 43; and a light chain variable region that comprises: (d) a LCDR1 of SEQ ID NO: 44, (e) a LCDR2 of SEQ ID NO:45, and (f) a LCDR3 of SEQ ID NO: 46.

The antibody or antigen-binding fragment, comprising:
(i) a heavy chain variable region (VH) comprising an amino acid sequence at least 90, 91, 92, 93, 94, 95, 96, 97, 98 or 99% identical to SEQ ID NO: 17, and a light chain variable region (VL) comprising an amino acid sequence at least 90, 91, 92, 93, 94, 95, 96, 97, 98 or 99% identical to SEQ ID NO: 18;
(ii) a heavy chain variable region (VH) comprising an amino acid sequence at least 90, 91, 92, 93, 94, 95, 96, 97, 98 or 99% identical to SEQ ID NO: 7, and a light chain variable region (VL) comprising an amino acid sequence at least 90, 91, 92, 93, 94, 95, 96, 97, 98 or 99% identical to SEQ ID NO: 8;
(iii) a heavy chain variable region (VH) comprising an amino acid sequence at least 90, 91, 92, 93, 94, 95, 96, 97, 98 or 99% identical to SEQ ID NO: 27, and a light chain variable region (VL) comprising an amino acid sequence at least 90, 91, 92, 93, 94, 95, 96, 97, 98 or 99% identical to SEQ ID NO: 28;
(iv) a heavy chain variable region (VH) comprising an amino acid sequence at least 90, 91, 92, 93, 94, 95, 96, 97, 98 or 99% identical to SEQ ID NO: 37, and a light chain variable region (VL) comprising an amino acid sequence at least 90, 91, 92, 93, 94, 95, 96, 97, 98 or 99% identical to SEQ ID NO: 38; or
(v) a heavy chain variable region (VH) comprising an amino acid sequence at least 90, 91, 92, 93, 94, 95, 96, 97, 98 or 99% identical to SEQ ID NO: 47, and a light chain variable region (VL) comprising an amino acid sequence at least 90, 91, 92, 93, 94, 95, 96, 97, 98 or 99% identical to SEQ ID NO: 48.

The antibody or antigen-binding fragment wherein one, two, three, four, five, six, seven, eight, nine, or ten amino acids within SEQ ID NO: 17, 18, 7, 8, 27, 28, 37, 38, 47 or 48 have been inserted, deleted or substituted.

The antibody or antigen-binding fragment that comprises:
(i) a heavy chain variable region (VH) that comprises SEQ ID NO:17, and a light chain variable region (VL) that comprises SEQ ID NO: 18;
(ii) a heavy chain variable region (VH) that comprises SEQ ID NO: 7, and a light chain variable region (VL) that comprises SEQ ID NO: 8;
(iii) a heavy chain variable region (VH) that comprises SEQ ID NO: 27, and a light chain variable region (VL) that comprises SEQ ID NO: 28;
(iv) a heavy chain variable region (VH) that comprises SEQ ID NO: 37, and a light chain variable region (VL) that comprises SEQ ID NO: 38; or
(v) a heavy chain variable region (VH) that comprises SEQ ID NO: 47, and a light chain variable region (VL) that comprises SEQ ID NO: 48.

The antibody or antigen-binding fragment, which is a monoclonal antibody, a chimeric antibody, a humanized antibody, a human engineered antibody, a single chain antibody (scFv), a Fab fragment, a Fab′ fragment, or a F(ab′)2 fragment.

The antibody or antigen-binding fragment, wherein the antibody or antigen-binding fragment thereof has antibody dependent cellular cytotoxicity (ADCC) or complement dependent cytotoxicity (CDC).

The antibody or antigen-binding fragment, wherein the antibody or antigen-binding fragment thereof has reduced glycosylation or no glycosylation or is hypofucosylated.

The antibody or antigen-binding fragment, wherein the antibody or antigen-binding fragment thereof comprises increased bisecting GlcNac structures.

A diagnostic reagent comprising the antibody or antigen-binding fragment thereof further comprising a detectable label.

The diagnostic reagent wherein the detectable label is selected from the group consisting of a radiolabel, a fluorophore, a chromophore, an imaging agent, and a metal ion.

A method of detecting DXd in a biological sample comprising the steps of:
(a) administering an antibody drug conjugate containing DXd to a subject in need thereof;
(b) contacting a biological sample taken from the subject with an anti-DXd antibody; and
(c) detecting a label.

The method wherein the biological sample is a tissue, a cell or a fluid.

The method wherein the fluid is serum, plasma or whole blood.

The method wherein the DXd antibody is directly labeled,

The method wherein a labeled secondary antibody contacts the anti-DXd antibody.

An isolated nucleic acid that encodes the antibody or antigen-binding fragment.

A vector comprising the nucleic acid.

A host cell comprising the nucleic acid or the vector.

A process for producing an antibody or antigen-binding fragment thereof comprising cultivating the host cell and recovering the antibody or antigen-binding fragment from the culture.

In one embodiment, the antibody or an antigen-binding fragment thereof comprises one or more complementarity determining regions (CDRs) comprising an amino acid sequence selected from a group consisting of SEQ ID NO: 11, SEQ ID NO: 12, SEQ ID NO: 13, SEQ ID NO: 14, SEQ ID NO: 15, SEQ ID NO: 16, SEQ ID NO: 1, SEQ ID NO: 2, SEQ ID NO: 3, SEQ ID NO: 4, SEQ ID NO: 5, SEQ ID NO: 6, SEQ ID NO: 21, SEQ ID NO: 22, SEQ ID NO: 23, SEQ ID NO: 24, SEQ ID NO:25, SEQ ID NO: 26, SEQ ID NO: 31, SEQ ID NO: 32, SEQ ID NO: 33, SEQ ID NO: 34, SEQ ID NO:35, SEQ ID NO: 36, SEQ ID NO: 41, SEQ ID NO: 42, SEQ ID NO: 43, SEQ ID NO: 44, SEQ ID NO:45, or SEQ ID NO: 46.

In another embodiment, the antibody or an antigen-binding fragment thereof comprises: a heavy chain variable region comprising one or more heavy chain complementarity determining regions (UCDRs) comprising an amino acid sequence selected from the group consisting of SEQ ID NO: 11, SEQ ID NO: 12, SEQ ID NO: 13, SEQ ID NO: 1, SEQ ID NO: 2, SEQ ID NO: 3, SEQ ID NO: 21, SEQ ID NO: 22, SEQ ID NO: 23, SEQ ID NO: 31, SEQ ID NO: 32 SEQ ID NO: 33, SEQ ID NO: 41, SEQ ID NO: 42, and SEQ ID NO: 43.

In another embodiment, the antibody or an antigen-binding fragment thereof comprises: a heavy chain variable region comprising three heavy chain complementarity determining regions (HCDRs) which are HCDR1 of SEQ ID NO: 11, HCDR2 of SEQ ID NO: 12 and HCDR3 of SEQ ID NO: 13; HCDR1 of SEQ ID NO: 1, HCDR2 of SEQ ID NO: 2; and HCDR3 of SEQ ID NO: 3; HCDR1 of SEQ ID NO: 21, HCDR2 of SEQ ID NO: 22; and HCDR3 of SEQ ID NO: 23; HCDR1 of SEQ ID NO: 31, HCDR2 of SEQ ID NO: 32, and HCDR3 of SEQ ID NO: 33; HCDR1 of SEQ ID NO: 41, HCDR2 of SEQ ID NO: 42; and HCDR3 of SEQ ID NO: 43.

In one embodiment, the antibody of the present disclosure or an antigen-binding fragment thereof comprises: (a) a heavy chain variable region comprising an amino acid sequence of SEQ ID NO: 17, SEQ ID NO: 7, SEQ ID NO: 27, SEQ ID NO: 37 or SEQ ID NO: 47 or an amino acid sequence at least 95%, 96%, 97%, 98% or 99% identical to any one of SEQ ID NO: 17, SEQ ID NO: 7, SEQ ID NO: 27, SEQ ID NO: 37 or SEQ ID NO: 47.

In another embodiment, the antibody of the present disclosure or an antigen-binding fragment thereof comprises: a heavy chain variable region comprising an amino acid sequence of SEQ ID NO: 17, SEQ ID NO: 7, SEQ ID NO: 27, SEQ ID NO: 37 or SEQ ID NO: 47, or an amino acid sequence with one, two, or three amino acid substitutions in the amino acid sequence of SEQ ID NO: 17, SEQ ID NO: 7, SEQ ID NO: 27, SEQ ID NO: 37 or SEQ ID NO: 47.

In one embodiment, the antibody of the present disclosure binds to DXd with a binding affinity (K_{D}) of from 1×10⁻⁶ M to 1×10⁻¹⁰ M. In another embodiment, the antibody of the present disclosure binds to DXd with a binding affinity (K_{D}) of about 1×10⁻⁶ M, about 1×10⁻⁷ M, about 1×10⁻⁸ M, about 1×10⁻⁹ M or about 1×10⁻¹⁰ M.

The present disclosure relates to isolated nucleic acids comprising nucleotide sequences encoding the amino acid sequence of the antibody or an antigen-binding fragment.

In one embodiment, the isolated nucleic acid comprises a VH nucleotide sequence of SEQ ID NO: 9, SEQ ID NO: 19, SEQ ID NO: 29, SEQ ID NO: 39, or SEQ ID NO: 49, or a nucleotide sequence comprising at least 95%, 96%, 97%, 98% or 99% identity to SEQ ID NO: 9, SEQ ID NO: 19, SEQ ID NO: 29, SEQ ID NO: 39, or SEQ ID NO: 49, and encodes the VH region of the antibody or an antigen-binding fragment of the present disclosure.

The present disclosure relates to isolated nucleic acids comprising nucleotide sequences encoding the amino acid sequence of the antibody or an antigen-binding fragment.

In one embodiment, the isolated nucleic acid comprises a VH nucleotide sequence of SEQ ID NO: 9, SEQ ID NO: 19, SEQ ID NO: 29, SEQ ID NO: 39, or SEQ ID NO: 49, paired with a VL nucleotide sequence of SEQ ID NO: 10, SEQ ID NO: 20, SEQ ID NO: 30, SEQ ID NO: 40, or SEQ ID NO: 50.

In another aspect, the present disclosure relates to a pharmaceutical composition or laboratory composition comprising the DXd antibody or antigen-binding fragment thereof.

In yet another aspect, the present disclosure relates to a method detecting at least one DXd molecule.

### BRIEF DESCRIPTION OF THE DRAWINGS

FIG. 1A shows a graph of initial immune titer profiles.

FIG. 1B shows a graph of initial immune titer profiles.

FIG. 1C is a table of initial immune titer profiles.

FIGS. 2A and 2B show that 65 total positive clones meet the screening criteria with free biotin-DXd competitive ELISA.

FIG. 3 depicts an ELISA of 5 anti-DXd antibodies on CRM197-DXd.

FIG. 4 shows an ELISA of 5 anti-DXd antibodies on biotin-DXd.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “DXd” refers to the molecular structures shown below and its derivatives.

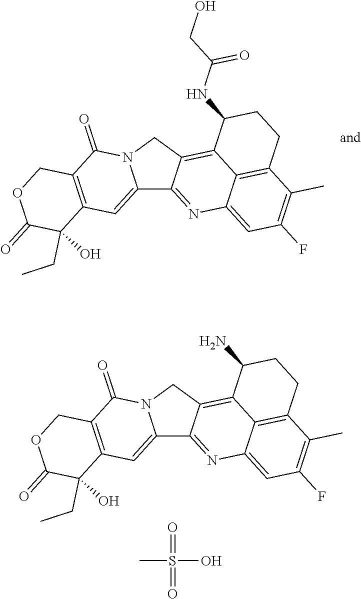

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR s), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contains a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

In some embodiments, the anti-DXd antibodies comprise at least one antigen-binding site, at least a variable region. In some embodiments, the anti-DXd antibodies comprise an antigen-binding fragment from an DXd antibody described herein. In some embodiments, the anti-DXd antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies comprising different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair comprising one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4).

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (ICT) numbering (Lefranc. N.-P., The Immunologist, 7, 132-136 (1999); Lefranc. N—P et al., Dev. Comp. Immunol, 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs are numbered 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof, specifically bind to a particular antigen at least ten (10) times greater when compared to the background level of binding and does not specifically bind in a significant amount to other antigens present in the sample.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to DXd. Common conservative substations of amino acids are well known in the art.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm which has been incorporated into the ALIGN program (version 2.0), using a PAM120 weight residue table, a gap length penalty of 12 and a gap penalty of 4 (E. Meyers and W. Miller, Comput. Appl. Biosci. 4: 11-17, (1988)). In addition, the percent identity between two amino acid sequences can be determined using the algorithm which has been incorporated into the GAP program in the GCG software package using either a BLOSLUM62 matrix or a PAM250 matrix, and a gap weight of 16, 14, 12, 10, 8, 6, or 4 and a length weight of 1, 2, 3, 4, 5, or 6 (Needleman and Wunsch, J. Mol. Biol. 48:444-453, (1970)).

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “label,” “detectable label” or “marker” is used herein to a molecule that generates a detectable signal or a molecule that acts on another molecule to generate a detectable signal. For example, a label can be a fluorescent molecule or other light emitting molecule, an enzyme, enzyme fragment or enzyme substrate or a radioactive substance. If the label is an enzyme, enzyme fragment or enzyme substrate, the detection of the label is the color or light developed in the reaction between the enzyme and a substrate (e.g., horseradish peroxidase (HWP)).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

### DETAILED DESCRIPTION

The present disclosure provides for antibodies, antigen-binding fragments or multivalent antibodies that specifically bind human DXd. Furthermore, the present disclosure provides antibodies that have desirable pharmacokinetic characteristics and other desirable attributes, and thus can be used for determining the concentration of DXd. The present disclosure further provides compositions comprising the antibodies or antigen binding fragments and methods of making and using such compositions for the quantitative or qualitative detection of DXd.

### Anti-DXd Antibodies

The present disclosure provides for antibodies or antigen-binding fragments thereof that specifically bind to DXd. Antibodies or antigen-binding fragments of the present disclosure include, but are not limited to, the antibodies or antigen-binding fragments thereof, generated as described, below.

The present disclosure provides antibodies or antigen-binding fragments that specifically bind to DXd, wherein said antibodies or antibody fragments (e.g., antigen-binding fragments) comprise a VH domain comprising an amino acid sequence of SEQ ID NO: 17, SEQ ID NO: 7, SEQ ID NO: 27, SEQ ID NO: 37 or SEQ ID NO: 47 (Table 1). The present disclosure also provides antibodies or antigen-binding fragments that specifically bind DXd, wherein said antibodies or antigen-binding fragments comprise a HCDR (heavy chain complementarity determining region) comprising an amino acid sequence of any one of the HCDRs listed in Table 1. In one aspect, the present disclosure provides antibodies or antigen-binding fragments that specifically bind to DXd, wherein said antibodies comprise (or alternatively, consist of) one, two, three, or more HCDRs comprising an amino acid sequence of any of the HCDRs listed in Table 1. In another aspect, the present disclosure provides antibodies or antigen-binding fragments that specifically bind to DXd, wherein said antibodies comprise (or alternatively, consist of) one, two, three, or more HCDRs and comprise (or alternatively, consist of) one, two, three or more LCDRs comprising an amino acid sequence of any of the LCDRs listed in Table 1

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 1. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% percent identity to the sequences described in Table 1. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that, encode the VH-domain and the VL domain of the antibodies that specifically bind to DXd. Such nucleic acid sequences can be optimized for expression in mammalian cells.

TABLE 1

| Antibody | SEQ ID NO | DOMAIN | SEQUENCE |
|---|---|---|---|
| 28C8 | SEQ ID NO: 1 | HCDR1 | SYAMN |
| | SEQ ID NO: 2 | HCDR2 | IIYDSGSTYYANWAKG |
| | SEQ ID NO: 3 | HCDR3 | GYLDI |
| | SEQ ID NO: 4 | LCDR1 | QSSKSVYNNKWLS |
| | SEQ ID NO: 5 | LCDR2 | GASTLAS |
| | SEQ ID NO: 6 | LCDR3 | AGGYSISSDNA |
| | SEQ ID NO: 7 | VH AA | METGLRWLLLVAVLKGVQCQSVEESGGRLVTP |
| | | | GTPLTLTCTVSGFSLSSYAMNWVRQAPGKGLEY |
| | | | IGIIYDSGSTYYANWAKGRFTISKTSTTVDLKITS |
| | | | PTTEDTATYFCARGYLDIWGPGTLVTVSS |
| | SEQ ID NO: 8 | VL AA | MDTRAPTQLLGLLLLWLPGARCAAVLTQTPSPV |
| | | | SAAVGGTVSISCQSSKSVYNNKWLSWLQQKPG |
| | | | QPPKLLIYGASTLASGVPSRFKGSGSGTEFTLTIS |
| | | | DVQCDDAATYYCAGGYSISSDNAFGGGTEVVV |
| | | | K |
| | SEQ ID NO: 9 | VH DNA | ATGGAGACTGGGCTGCGCTGGCTTCTCCTGGT |
| | | | CGCTGTGCTCAAAGGTGTCCAGTGTCAGTCG |
| | | | GTGGAGGAGTCCGGGGGTCGCCTGGTCACGC |
| | | | CTGGGACACCCCTGACACTCACCTGCACAGT |
| | | | CTCTGGATTCTCCCTCAGTAGCTATGCAATGAA |
| | | | CTGGGTCCGCCAGGCTCCAGGAAAGGGGCTG |
| | | | GAATACATCGGAATTATTTATGATAGTGGTAGC |
| | | | ACATACTACGCGAACTGGGCGAAAGGCCGATT |
| | | | CACCATCTCCAAAACCTCGACCACGGTGGATC |
| | | | TGAAAATCACCAGTCCGACAACCGAGGACAC |
| | | | GGCCACCTATTTCTGTGCCAGAGGTTATTTAG |
| | | | ACATCTGGGGCCCAGGCACCCTGGTCACCGTC |
| | | | TCCTCA |
| | SEQ ID NO: 10 | VL DNA | ATGGACACGAGGGCCCCCACTCAACTGCTGG |
| | | | GGCTCCTGCTGCTCTGGCTCCCAGGTGCCAGA |
| | | | TGTGCCGCCGTGCTGACCCAGACTCCATCTCC |
| | | | CGTGTCTGCAGCTGTGGGAGGCACAGTCAGC |
| | | | ATCAGTTGCCAGTCCAGTAAGAGTGTTTATAA |
| | | | TAACAAGTGGCTATCCTGGCTTCAGCAGAAAC |
| | | | CAGGGCAGCCTCCCAAGCTCCTGATCTATGGT |
| | | | GCATCCACTCTGGCATCTGGGGTCCCATCGCG |
| | | | GTTCAAAGGCAGTGGATCTGGGACAGAGTTC |
| | | | ACTCTCACCATCAGCGACGTGCAGTGTGACG |
| | | | ATGCTGCCACTTACTACTGTGCGGGCGGTTATA |
| | | | GTATTAGTAGTGATAATGCTTTCGGCGGAGGG |
| | | | ACCGAGGTGGTGGTCAAA |
| 29D2 | SEQ ID NO: 11 | HCDR1 | SYAMN |
| | SEQ ID NO: 12 | HCDR2 | IIYDSGTTYYASWAKG |
| | SEQ ID NO: 13 | HCDR3 | AALDI |
| | SEQ ID NO: 14 | LCDR1 | QASKSVYNNNWLS |
| | SEQ ID NO: 15 | LCDR2 | GASTLAS |
| | SEQ ID NO: 16 | LCDR3 | AGGYGSSSDNA |
| | SEQ ID NO: 17 | VH AA | METGLRWLLLVAVLKGVQCQSVEESGGRLVTP |
| | | | GTPLTLTCTASGFSLSSYAMNWVRQAPGKGLEY |
| | | | IGIIYDSGTTYYASWAKGRFTISKTSTTVDLKITS |
| | | | PTTEDTATYFCARAALDIWGPGTLVTVSS |
| | SEQ ID NO: 18 | VL AA | MDTRAPTQLLGLLLLWLPGATFAAVLTQTPSPV |
| | | | SAAVGGTVSISCQASKSVYNNNWLSWVQQTPG |
| | | | QPPKLLIYGASTLASGVPSRFKGSGSGTQFTLTIS |
| | | | DVQCDDAATYYCAGGYGSSSDNAFGGGTEVV |
| | | | VE |
| | SEQ ID NO: 19 | VH DNA | ATGGAGACTGGGCTGCGCTGGCTTCTCCTGGT |
| | | | CGCTGTGCTCAAAGGTGTCCAGTGTCAGTCG |
| | | | GTGGAGGAGTCCGGGGGTCGCCTGGTCACGC |
| | | | CTGGGACACCCCTGACACTCACCTGCACAGC |
| | | | CTCTGGATTCTCCCTCAGTAGCTATGCAATGAA |
| | | | CTGGGTCCGCCAGGCTCCAGGGAAGGGGCTG |
| | | | GAATACATCGGAATCATTTATGATAGTGGCACC |
| | | | ACATACTACGCGAGCTGGGCGAAAGGCCGATT |
| | | | CACCATCTCCAAAACCTCGACCACGGTGGATC |
| | | | TGAAAATCACCAGTCCGACAACCGAAGACAC |
| | | | GGCCACTTATTTCTGTGCCAGAGCAGCATTGG |
| | | | ACATCTGGGGCCCAGGCACCCTGGTCACCGTC |
| | | | TCCTCA |
| | SEQ ID NO: 20 | VL DNA | ATGGACACGAGGGCCCCCACTCAGCTGCTGG |
| | | | GGCTCCTGCTGCTCTGGCTCCCAGGTGCCACA |
| | | | TTTGCCGCCGTGCTGACCCAGACTCCGTCTCC |
| | | | CGTGTCTGCAGCTGTGGGAGGCACAGTCAGC |
| | | | ATCAGTTGCCAGGCCAGTAAGAGTGTTTATAA |
| | | | TAACAACTGGTTATCCTGGGTTCAGCAGACAC |
| | | | CAGGGCAGCCTCCCAAGCTCCTGATCTATGGT |
| | | | GCATCCACTCTGGCATCTGGGGTCCCATCGCG |
| | | | GTTCAAAGGCAGTGGATCTGGGACACAGTTC |
| | | | ACTCTCACCATTAGCGACGTGCAGTGTGACGA |
| | | | TGCTGCCACTTACTACTGTGCAGGCGGTTATG |
| | | | GTAGTAGTAGTGATAATGCTTTCGGCGGAGGG |
| | | | ACCGAGGTGGTGGTCGAA |
| 35D6 | SEQ ID NO: 21 | HCDR1 | SHWMS |
| | SEQ ID NO: 22 | HCDR2 | IISSSGSTDYVSWAKG |
| | SEQ ID NO: 23 | HCDR3 | DLGYSTNI |
| | SEQ ID NO: 24 | LCDR1 | QASQSIGSYLA |
| | SEQ ID NO: 25 | LCDR2 | DASTLAS |
| | SEQ ID NO: 26 | LCDR3 | QQGYSSSGVDNA |
| | SEQ ID NO: 27 | VH AA | METGLRWLLLVAVLKGVQCQSVEESGGRLVTP |
| | | | GTPLTLTCTVSGFSLSSHWMSWVRQAPGKGLE |
| | | | WIGIISSSGSTDYVSWAKGRFTISKTSTTVDLKIT |
| | | | SPTTEDTATYFCARDLGYSTNIWGPGTLVTVSL |
| | SEQ ID NO: 28 | VL AA | MDTRAPTQLLGLLLLWLPGARCAYDMTQTPAS |
| | | | VEVTVGGTVTIKCQASQSIGSYLAWYQQKPGQ |
| | | | RPKLLIYDASTLASGVSSRFKGSGSGTQFTLTISG |
| | | | VECADAATYYCQQGYSSSGVDNAFGGGTEVV |
| | | | VK |
| | SEQ ID NO: 29 | VH DNA | ATGGAGACTGGGCTGCGCTGGCTTCTCCTGGT |
| | | | CGCTGTGCTCAAAGGTGTCCAGTGTCAGTCG |
| | | | GTGGAGGAGTCCGGGGGTCGCCTGGTCACGC |
| | | | CTGGGACACCCCTGACACTCACCTGCACAGT |
| | | | CTCTGGATTCTCCCTGAGTAGCCATTGGATGA |
| | | | GCTGGGTCCGCCAGGCTCCAGGGAAGGGGCT |
| | | | GGAATGGATCGGAATCATCAGTAGTAGTGGTA |
| | | | GTACGGACTACGTGAGCTGGGCGAAAGGCCG |
| | | | ATTCACCATCTCCAAAACCTCGACCACGGTGG |
| | | | ATCTGAAAATCACCAGTCCGACAACCGAGGA |
| | | | CACGGCCACCTATTTCTGTGCCAGAGATCTGG |
| | | | GTTATAGTACTAACATCTGGGGCCCAGGCACC |
| | | | CTGGTCACCGTCTCCTTA |
| | SEQ ID NO: 30 | VL DNA | ATGGACACGAGGGCCCCCACTCAGCTGCTGG |
| | | | GGCTCCTGCTGCTCTGGCTCCCAGGTGCCAGA |
| | | | TGTGCCTATGATATGACCCAGACTCCAGCCTCT |
| | | | GTGGAGGTAACTGTGGGAGGCACAGTCACCA |
| | | | TCAAGTGCCAGGCCAGTCAGAGCATTGGTAG |
| | | | CTACTTAGCCTGGTATCAGCAGAAACCAGGAC |
| | | | AGCGTCCCAAGCTCCTGATCTACGATGCATCC |
| | | | ACTCTGGCATCTGGGGTCTCATCGCGGTTCAA |
| | | | AGGCAGTGGATCTGGGACACAGTTCACTCTC |
| | | | ACCATCAGCGGCGTGGAGTGTGCCGATGCTGC |
| | | | CACTTACTACTGTCAACAGGGTTATAGTAGTA |
| | | | GTGGTGTTGATAATGCTTTCGGCGGAGGGACC |
| | | | GAGGTGGTGGTCAAA |
| 76G4 | SEQ ID NO: 31 | HCDR1 | SYYMF |
| | SEQ ID NO: 32 | HCDR2 | YIGDGGYTSYATWAKG |
| | SEQ ID NO: 33 | HCDR3 | DGFTSGSI |
| | SEQ ID NO: 34 | LCDR1 | QSSKNVYNNNWLS |
| | SEQ ID NO: 35 | LCDR2 | GTSTLAS |
| | SEQ ID NO: 36 | LCDR3 | AGGYSSSSDNG |
| | SEQ ID NO: 37 | VH AA | METGLRWLLLVAVLKGVQCQSVEESGGRLVTP |
| | | | GTPLTLTCTASGFSLSSYYMFWVRQAPGKGLEY |
| | | | IGYIGDGGYTSYATWAKGRFTISKTSSTTVDLKI |
| | | | TSPTTEDTATYFCARDGFTSGSIWGPGTLVTVSS |
| | SEQ ID NO: 38 | VL AA | MDTRAPTQLLGLLLLWLPGATFAAVLTQTPSPV |
| | | | STAVGGTVTISCQSSKNVYNNNWLSWFQQKPG |
| | | | QPPKLLIYGTSTLASGVPSRFKGSGSGTQFTLTIS |
| | | | DVQCDDAATYYCAGGYSSSSDNGFGAGTEVVV |
| | | | K |
| | SEQ ID NO: 39 | VH DNA | ATGGAGACTGGGCTGCGCTGGCTTCTCCTGGT |
| | | | CGCTGTGCTCAAAGGTGTCCAGTGTCAGTCG |
| | | | GTGGAGGAGTCCGGGGGTCGCCTGGTCACGC |
| | | | CTGGGACACCCCTGACACTCACCTGCACAGC |
| | | | CTCTGGATTCTCCCTCAGTAGCTACTACATGTT |
| | | | CTGGGTCCGCCAGGCTCCAGGGAAGGGGCTA |
| | | | GAATACATCGGATACATTGGTGATGGTGGTTAC |
| | | | ACATCCTACGCGACCTGGGCAAAAGGCCGATT |
| | | | CACCATCTCCAAAACCTCGTCGACCACGGTGG |
| | | | ATCTGAAAATCACCAGTCCGACAACCGAGGA |
| | | | CACGGCCACCTATTTCTGTGCCAGAGACGGTT |
| | | | TTACTTCTGGTAGCATTTGGGGCCCAGGCACC |
| | | | CTGGTCACCGTTTCCTCA |
| | SEQ ID NO: 40 | VL DNA | ATGGACACGAGGGCCCCCACTCAGCTGCTGG |
| | | | GGCTCCTGCTGCTCTGGCTCCCAGGTGCCACA |
| | | | TTTGCCGCCGTGCTGACCCAGACTCCATCTCC |
| | | | CGTGTCTACAGCTGTGGGAGGCACAGTCACC |
| | | | ATCAGTTGCCAGTCCAGTAAGAATGTTTATAAT |
| | | | AACAACTGGTTATCCTGGTTTCAGCAGAAACC |
| | | | AGGGCAGCCTCCCAAGCTCCTCATCTATGGTA |
| | | | CATCCACGCTGGCGTCTGGGGTCCCATCGCGG |
| | | | TTCAAAGGCAGTGGATCTGGGACACAGTTCA |
| | | | CTCTCACCATCAGCGACGTGCAGTGTGACGAT |
| | | | GCTGCCACTTACTACTGTGCAGGCGGTTATAG |
| | | | TAGTAGTAGTGATAATGGTTTCGGCGCAGGGA |
| | | | CCGAGGTGGTGGTCAAA |
| 77G10 | SEQ ID NO: 41 | HCDR1 | SYYMS |
| | SEQ ID NO: 42 | HCDR2 | IISSSGSTDYASWAKG |
| | SEQ ID NO: 43 | HCDR3 | GWLDL |
| | SEQ ID NO: 44 | LCDR1 | QSSKSVYNNNWLS |
| | SEQ ID NO: 45 | LCDR2 | GASTLAS |
| | SEQ ID NO: 46 | LCDR3 | AGGYSSSTDNA |
| | SEQ ID NO: 47 | VH AA | METGLRWLLLVAVLKGVQCQSLEESGGRLVTPG |
| | | | TPLTLTCTASGFSLSSYYMSWVRQAPGKGLEYI |
| | | | GIISSSGSTDYASWAKGRFTISKTSTTVDLKITSP |
| | | | TTEDTATYFCARGWLDLWGQGTLVTVSS |
| | SEQ ID NO: 48 | VL AA | MDTRAPTQLLGLLLLWLPGATFAAVLTQTPSPV |
| | | | SAAVGGTVSISCQSSKSVYNNNWLSWLQQKPG |
| | | | QPPKLLIHGASTLASGVPSRFKGSGSGTQFTLTIS |
| | | | DVQCDDAATYYCAGGYSSSTDNAFGGGTEVV |
| | | | VK |
| | SEQ ID NO: 49 | VH DNA | ATGGAGACTGGGCTGCGCTGGCTTCTCCTGGT |
| | | | CGCTGTGCTCAAAGGTGTCCAGTGTCAGTCGC |
| | | | TGGAGGAGTCCGGGGGTCGCCTGGTCACGCC |
| | | | TGGGACACCCCTGACACTCACCTGCACAGCC |
| | | | TCTGGATTCTCCCTCAGTAGTTACTACATGAGC |
| | | | TGGGTCCGCCAGGCTCCAGGGAAGGGGCTGG |
| | | | AATACATCGGAATCATTAGTAGTAGTGGTTCCA |
| | | | CGGACTACGCGAGCTGGGCGAAAGGCCGATT |
| | | | CACCATCTCCAAAACCTCGACCACGGTGGATC |
| | | | TGAAAATCACCAGTCCGACAACCGAGGACAC |
| | | | GGCCACCTATTTCTGTGCCAGAGGCTGGTTGG |
| | | | ATCTCTGGGGCCAGGGCACCCTGGTCACCGTC |
| | | | TCTTCA |
| | SEQ ID NO: 50 | VL DNA | ATGGACACGAGGGCCCCCACTCAGCTGCTGG |
| | | | GGCTCCTGCTGCTCTGGCTCCCAGGTGCCACA |
| | | | TTTGCCGCCGTGCTGACCCAGACTCCATCTCC |
| | | | CGTGTCTGCAGCTGTGGGAGGCACAGTCAGC |
| | | | ATCAGTTGCCAGTCCAGTAAGAGTGTTTATAA |
| | | | TAACAACTGGTTATCCTGGCTTCAGCAGAAAC |
| | | | CAGGGCAGCCTCCCAAGCTCCTGATCCATGGT |
| | | | GCATCCACTCTGGCATCTGGGGTCCCATCGCG |
| | | | GTTCAAAGGCAGTGGATCTGGGACACAGTTC |
| | | | ACTCTCACCATCAGCGACGTGCAGTGTGACG |
| | | | ATGCTGCCACTTACTACTGTGCAGGCGGTTATA |
| | | | GTAGTAGTACTGATAATGCTTTCGGCGGAGGG |
| | | | ACCGAGGTGGTGGTCAAA |

### DXd Immunogen Preparation

Carrier Protein Conjugation with [DXd-PEG4-S]₂

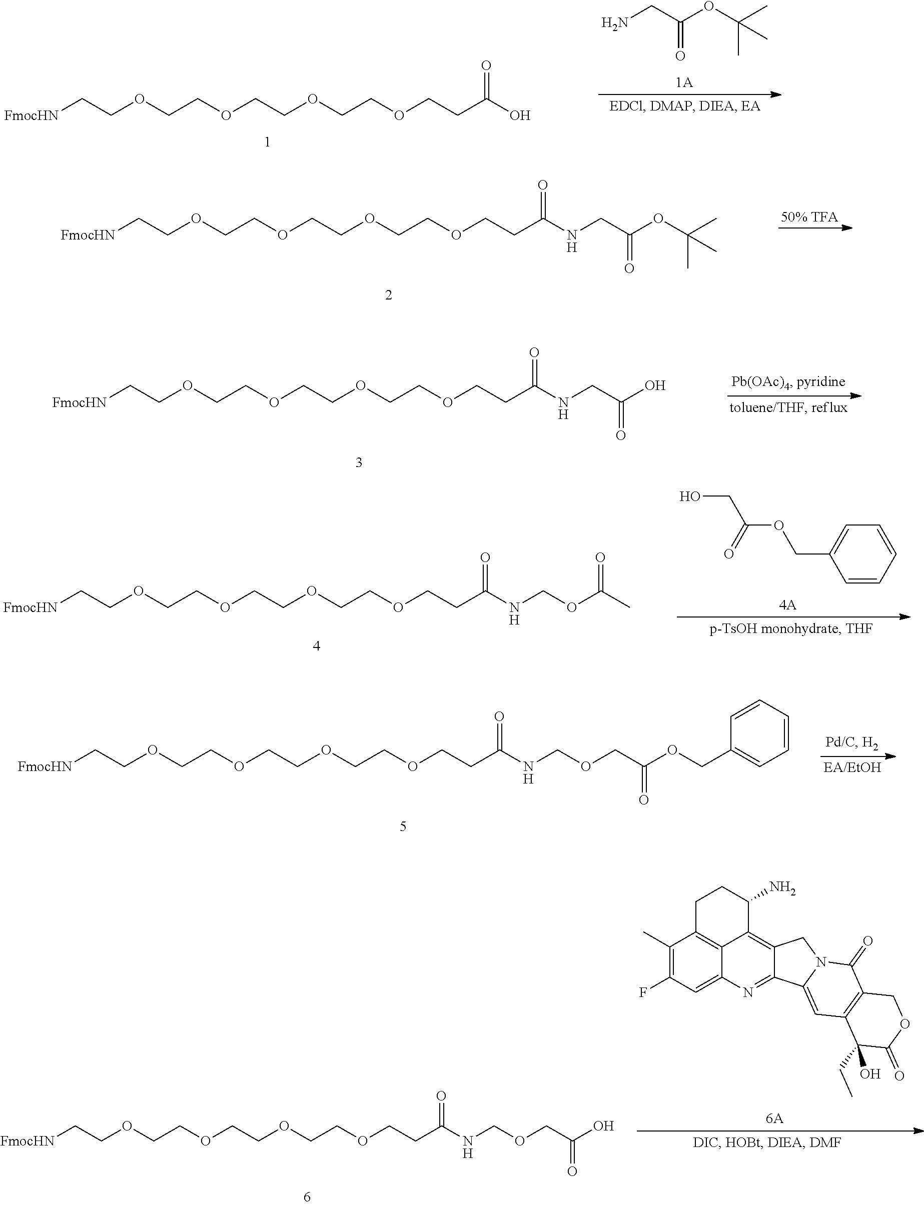

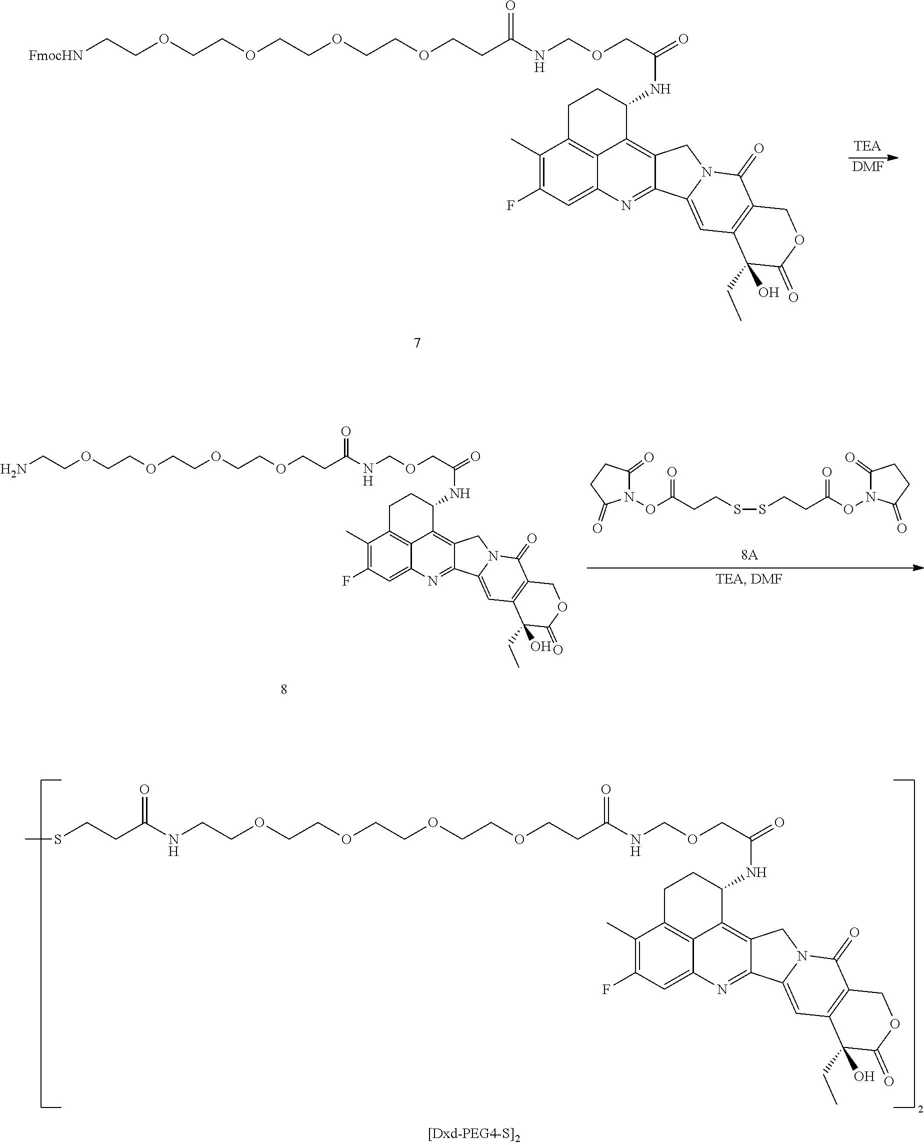

### General Procedure for Preparation of Compound 2

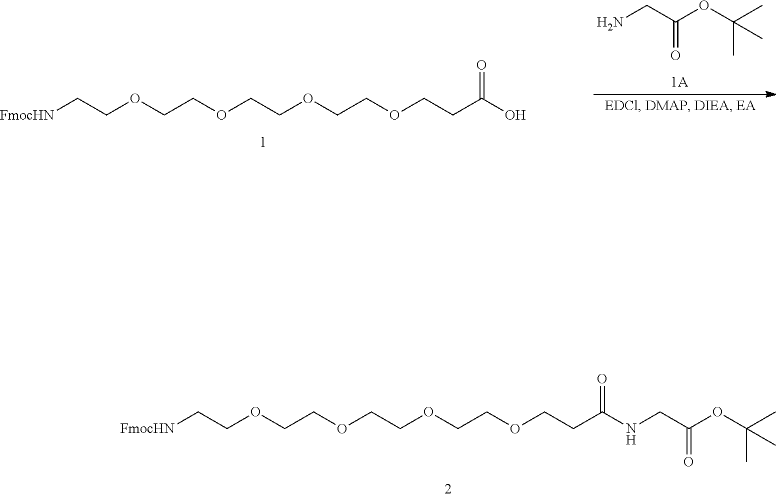

To a solution of compound 1 (5.0 g, 10.2 mmol, 1 eq) and compound IA (2.0 g, 15.3 mmol, 1.5 eq) in EA (50 mL) was added DIEA (3.98 g, 30.7 mmol, 5.36 mL, 3 eq), HOBt (1.52 g, 11.2 mmol, 1.1 eq) and EDCI (3.93 g, 20.5 mmol, 2 eq), the reaction mixture was stirred at 25° C. for 2 hours. LC-MS showed compound 1 was consumed completely and one main peak with desired mass was detected. The reaction was diluted with LA (50 mL) and washed with 1 M HCl (50 mL), the organic phase was separated, and the solution phase was extracted with EA (50 mL) The combined organic layers were washed with brine (50 mL), dried over anhydrous Na₂SO₄, filtered and concentrated under reduced pressure to give a residue. The residue was purified by column chromatography (SiO₂, DCM/MeOH=100/1 to 50/1) to give compound 2 (6.0 g, 97.4% yield) as a yellow oil. Compound 2 LCMS [MH]*: 601.2

### General Procedure for Preparation of Compound 3

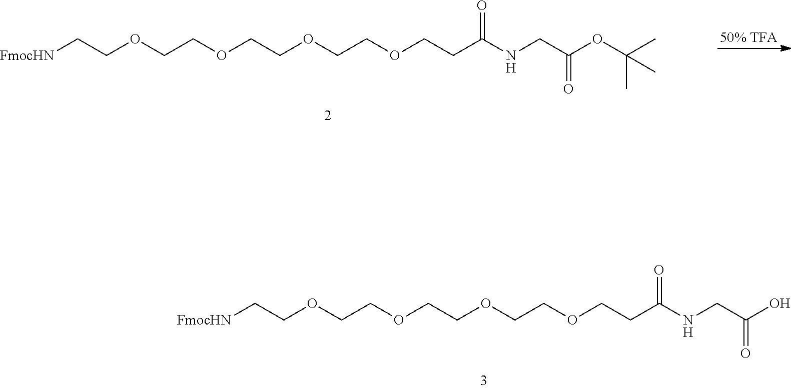

To a solution of compound 2 (6.0 g, 9.99 mmol, 1 eq) in DCM (30 mL) was added TFA (30 mL). The mixture was stirred at 25° C. for 2 hours. TLC (DCM:MeOH=10:1, R_{f}=0.3) showed compound 2 was consumed completely. The reaction mixture was concentrated under reduced pressure to remove solvent. The crude product was used into the next step without further purification to give compound 3 (4.5 g, 82.7% yield) as a yellow oil. Compound 3 LCMS [MH]: 545.2

### General Procedure for Preparation of Compound 4

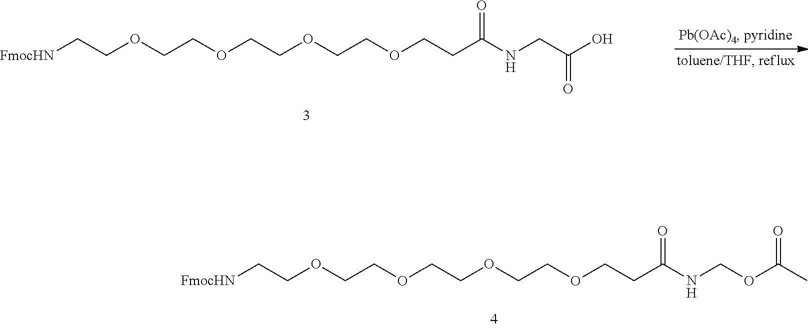

A mixture of compound 3 (4.5 g, 8.26 mmol, 1 eq) in toluene (45 mL) and THF (135 mL) was added to pyridine (6.54 g, 82.6 mmol, 6.67 mL, 10 eg), then anhydrous Pb(OAc)₄ (9.16 g, 20.6 mmol, 2.5 eq) was added, stirred at reflux temperature (external temp. 120° C.) for 2 hours under N₂ atmosphere. TLC (PE:EA=1:1, R_{f}=0.7) showed the reaction was completed. After the reaction solution was cooled to room temperature, the insoluble material was removed by filtration, and the filtrate was concentrated under reduce pressure. The obtained residue was dissolved in ethyl acetate, washed with water and saturated brine, and then the organic layer was dried over anhydrous Na₂SO₄. The solvent was removed under reduced pressure. The crude product was used into the next step without further purification to give compound 4 (5.0 g, crude) as a yellow oil. Compound 4 LCMS [MH]⁺: 581.2, retention time on LC: 0.915 min

### General Procedure for Preparation of Compound 5

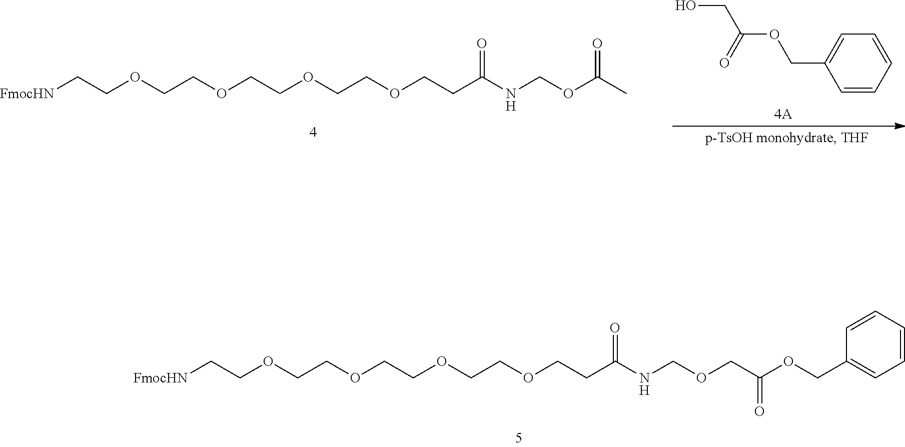

To a mixture of compound 4 (5 g, 8.95 mmol, 1 eq) and compound 4A (4.46 g, 26.85 mmol, 3.81 mL, 3 eq) in THF (50 mL) was added 4-methylbenzenesulfonic acid monohydrate (77.07 mg, 447.53 umol, 0.05 eq), then the mixture was stirred at 25° C. for 2 hours under N₂ atmosphere. TLC (PE:EA=1:1, R_{f}=0.2) showed the reaction was completed. The mixture was diluted with NaHCO₃ (50 mL) and extracted with EtOAc (50 mL). The combined organic layers were dried with Na₂SO₄, filtered and concentrated under reduced pressure to give a residue. The residue was purified by prep-HPLC (neutral condition) to give compound 5 (2.5 g, 42.0% yield) as a white solid. Compound 5, poor LCMS signal but fragment species was observed [FmocNH-PEG4-CO—NH—CH₂]⁺ was observed: 499.2, retention time on LC: 0.980 min.

### General Procedure for Preparation of Compound 6

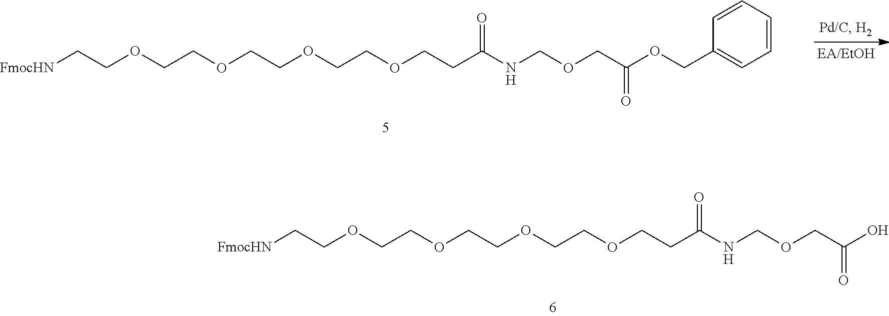

To a mixture of compound 5 (2.5 g, 3.76 mmol, 1 eq) in ethanol (100 mL) and ethyl acetate (300 mL) was added dry Pd/C (400 mg), then the reaction mixture was stirred at 25° C. for 2 hours under H₂ atmosphere (15 psi). TLC (DCM:MeOH=10:1, R_{f}=0.2) showed compound 5 was consumed and one new spot was formed. The reaction mixture was filtered through siliceous earth, and the filtrate was concentrated under reduced pressure to give compound 6 (2.1 g, crude) as a faint yellow solid. Compound 6, poor LCMS signal but fragment species was observed [FmocNH-PEG4-CO—NH—CH₂]⁺ was observed: 499.2, retention time on LC: 0.889 min

### General Procedure for Preparation of Compound 7

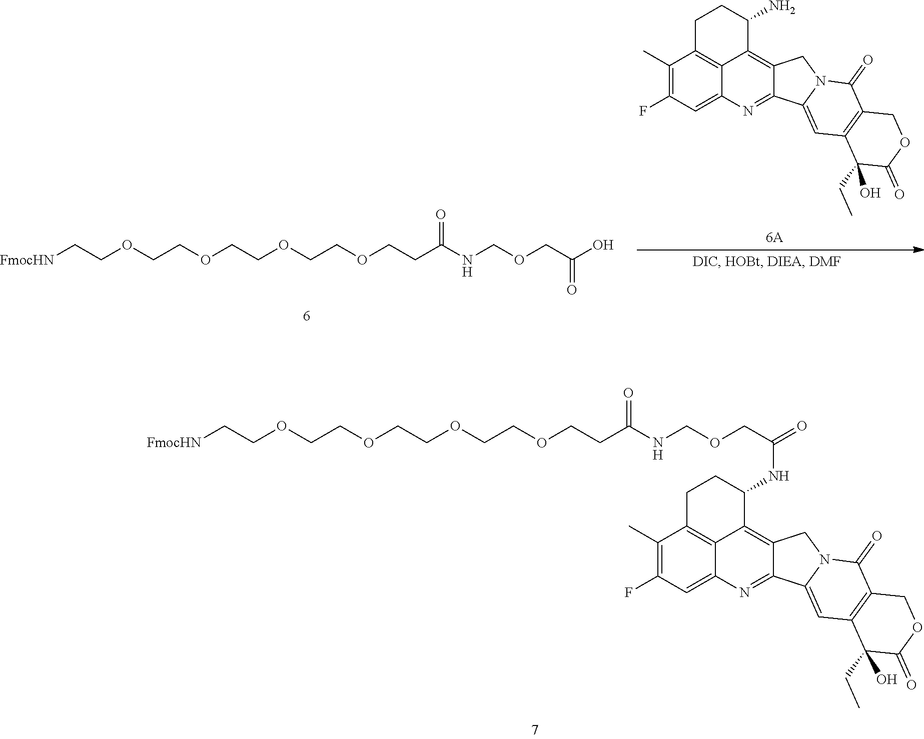

To a solution of compound 6 (150 mg, 261.04 umol, 1.3 eq) and compound 6A in DMF (1 mL) was added HOBt (29.85 mg, 220.88 umol, 1.1 eq), DIC (101.36 mg, 803.21 umol, 124.37 uL, 4 eq) and DIEA (25.95 mg, 200.80 umol, 34.98 uL, 1 eq). The mixture was stirred at 40° C. for 2 hours. LC-MS showed compound 6A was consumed completely and one main peak with desired mass was detected. The mixture was purified by prep-HPLC.

### General Procedure for Preparation of Compound 8

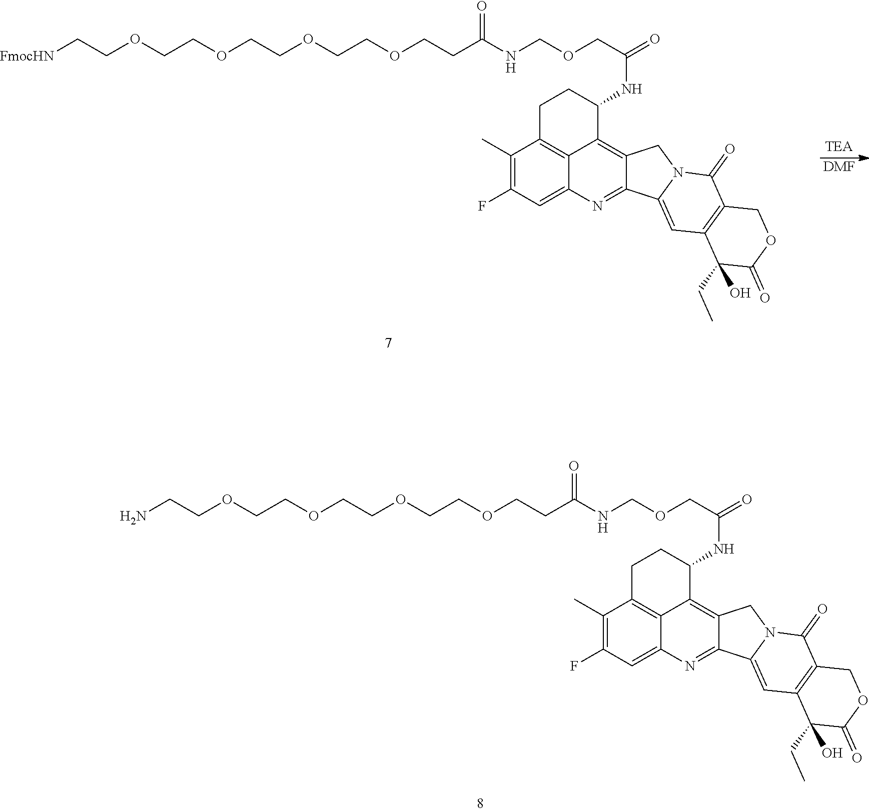

To a solution of compound 7 (190 mg, 191 umol, 1 eq) in DMF (800 uL) was added TEA (200 uL). The mixture was stirred at 25° C. for 3 hours. LC-MS showed the reaction was completed. The crude product in the DMF solution was used for the next step without further work up and purification. Compound 8 [MH]: 770.4

### General Procedure for Preparation of [DXd-PEG4-S]₂

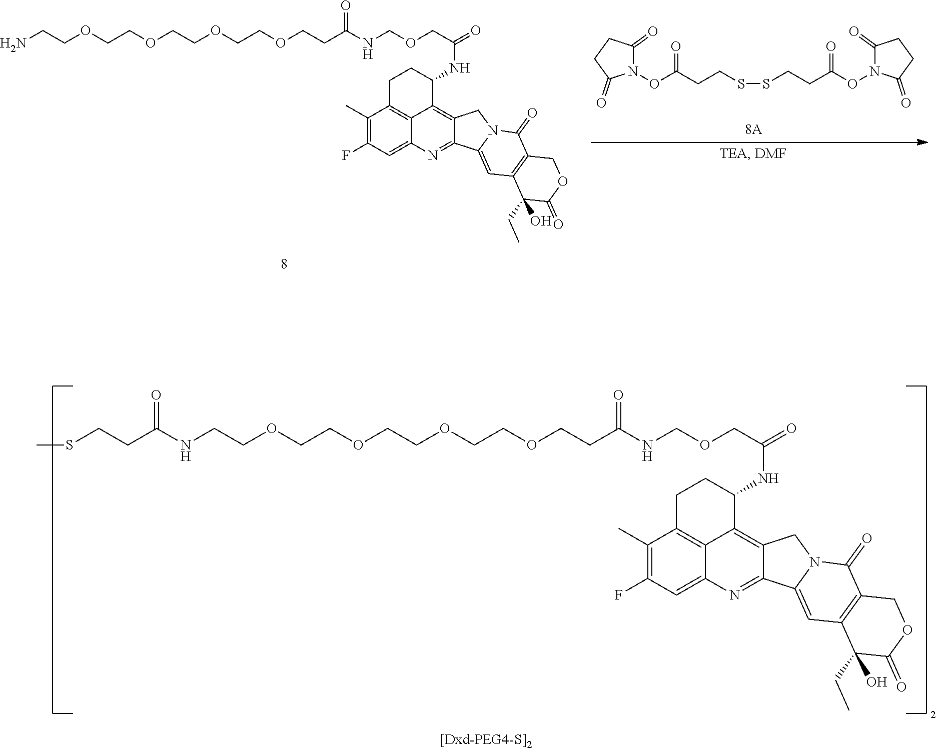

To a solution of crude compound 8 (191 umol) in DMF was added compound 8A (38.7 mg, 95.7 umol), then the reaction mixture was stirred at 25° C. for 0.5 hour. LC-MS showed the reaction was completed. The mixture was directly purified by prep-HPLC (basic condition) to give [DXd-PEG4-SH]₂ (28 mg, 97.0% purity, 17% yield) as a white solid. Compound [DXd-PEG4-S]₂ [MH]⁺: 1713.5600

Carrier Protein Conjugation with [DXd-PEG4-S]₂

TABLE 2
Reduction condition for linker-drug dimer

| LD dimer | TCEP/LD | | Temp. | Time |
|---|---|---|---|---|
| Conc. (mM) | ratio | DMA % | (° C.) | (h) |
| 2.0 | 1.1 | 50 | 37 | 2 |

For the reduction, DMA, 10 mM LD dimer, dH₂O and 100 mM TCEP were added in such order and mixed well. The reaction concentration of LD dimer was 2 mM. The reaction mixture was incubated on Thermomixer (400 rpm) at 37° C. for 2 hours. After the reaction, the sample was diluted 200-fold with 10% DMA for RP-HPLC detection. The reduction efficiency of [DXd-PEG4-S]₂ was 93.01%. Reduced product was stored at −80° C.

KLH Conjugation with HS-PEG4-DXd

Maleimide activated KLH was purchased from Sigma Aldrich (product no.: 10892). There are 240 maleimide moieties on one KLH molecule. [HS-PEG4-DXd] in DMA solution (2-5 mM, equivalent toward KLH: 3.5-6) was added into KLH in PBS buffer (concentration range: 3-4 mg/mL). DMA was less than 20 v/v % (between 10-20 v/v %) and KLH concentration was 2.5-3 mg/mL in the final reaction mixture. The reaction was left at 22° C. for 2 hours. KLH-S-PEG-DXd conjugate was then buffer exchanged into its storage buffer, 20 mM succinic buffer (pH 6.0). Under the above conditions, 50-85% maleimide capped with S-PEG4-DXd KLH conjugate was obtained.

BSA Conjugation with HS-PEG4-DXd

Bovine serum albumin (BSA) was purchased form Sigma Aldrich (product no: A7030). BSA in conjugation buffer (10 mM PBS, pH 7.4, 40 mM sucrose) was mixed with linker solution (linker: N—ε-maleimidocaproyl-oxysulfo-succinimide ester, Sulfo-EMCS, purchased from Thermo Scientific, product no: 22307) in conjugation buffer (50-60 eq.). Concentration of BSA in reaction solution was 3-6 mg/mL. The reaction was left at 22° C. for 1 hour. The reaction mixture was treated to a spin desalting column, pre-equilibrated with citric buffer (100 mM). The resulting linker-BSA conjugate was then added to the reduced HS-PEG4-DXd solution (in DMA, freshly prepared, 20-30 eq.) and the reaction was left at 4 C for an overnight period. The concentration of linker-BSA conjugate was 2-3 mg/mL and DMA was 10 v/v %. The resulting BSA-S-PEG-DXd conjugate was then dialyzed against storage buffer (20 mM succinic buffer, pH 6.0). Under above conditions, BSA-S-PEG4-DXd conjugate with average Linker-BSA-Ratio 15-25 and an average Drug-BSA-Ratio of 8-11 was obtained.

CRM197 Conjugation with HS-PEG4-DXd

CRM is a genetically modified (non-toxic) mutant of diphtheria toxin. CRM197 was conjugated with linker, Sulfo-EMCS and drug, HS-PEG4-DXd by the similar procedure described in BSA conjugation with HS-PEG4-DXd (Linker eq.: 20-40, drug eq: 7-8 eq). Under such condition, CRM197-S-PEG4-DXd conjugate with average Linker-CRM197-Ratio of 10-14 and average Drug-CRM197-Ratio of 4-5 was obtained.

### Alteration of the Fe Region

In yet other aspects, the Fe region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (Shields et al., J. Biol. Chem. 276:6591-6604, 2001).

In still another aspect, the glycosylation of the DXd antibody or antigen binding fragment is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody comprising reduced amounts of fucosyl residues or an antibody comprising increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lec13 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)—N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore et al., 2010 MAbs, 2:181-189). However, natural IgG4 was found less stable in stress conditions such as in acidic buffer or under increasing temperature (Angal, 1993 Mol Immunol, 30:105-108; DallAcqua et al, 1998 Biochemistry, 37:9266-9273; Aalberse et al., 2002 Immunol, 105:9-19). Reduced ADCC can be achieved by operably linking the antibody to an IgG4 Fe engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel et al., 1991 Proc. Natl. Acad. Sci. USA, 88:9036-9040; Mukherjee et al., 1995 FASEB J, 9:115-119; Armour et al., 1999 Eur J Immunol, 29:2613-2624; Clynes et al, 2000 Nature Medicine, 6:443-446; Arnold, 2007 Annu Rev immunol, 25:21-50). Furthermore, some rarely occurring IgG4 isoforms in human population can also elicit different physicochemical properties (Brusco et al., 1998 Eur J Immunogenet, 25:349-55; Aalberse et al., 2002 Immunol, 105:9-19). To generate DXd antibodies with low ADCC and CDC but with good stability, it is possible to modify the hinge and Fe region of human IgG4 and introduce a number of alterations. These modified IgG4 Fc molecules can be found in SEQ ID NOs: 83-88, U.S. Pat. No. 8,735,553 to Li et al.

### DXd Antibody Production

Anti-DXd antibodies, antigen-binding fragments and multispecific antibodies can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93% 94%, 95%, 96%, 97%, 98%, 99%, or 100% nucleic acid sequence identity with polynucleotides of SEQ ID NO: 9 and SEQ ID NO: 10; SEQ ID NO: 19 and SEQ ID NO: 20; SEQ ID NO: 29 and SEQ ID NO: 30; SEQ ID NO: 39; and SEQ ID NO: 40 or SEQ ID NO: 49 and SEQ ID NO: 50.

The polynucleotides of the present disclosure can encode a variable region sequence of an anti-DXd antibody. They can also encode both a variable region and a constant region of the antibody. Some of the polynucleotide sequences encode a polypeptide that comprises variable regions of both the heavy chain and the light chain of one of the exemplified anti-DXd antibodies.

Also provided in the present disclosure are expression vectors and host cells for producing the anti-DXd antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-DXd antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-DXd antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-DXd antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-DXd antibodies. Insect cells in combination with baculovirus vectors can also be used.

In other aspects, mammalian host cells are used to express and produce the anti-DXd polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of DXd. In one aspect, the antibodies or antigen-binding fragments are useful for detecting the presence of DXd in a biological sample. The term “detecting” as used herein includes quantitative or qualitative detection. In certain aspects, a biological sample comprises cells, tissue, urine, sputum, serum or blood samples. In other aspects, such tissues include normal and/or cancerous tissues or cells that contain DXd at higher levels relative to other tissues.

In one aspect, the present disclosure provides a method of detecting the presence of DXd in a biological sample. In certain aspects, the method comprises contacting the biological sample with an anti-DXd antibody under conditions permissive for binding of the antibody to the antigen and detecting whether a complex is formed between the antibody and the antigen. The biological sample can include, without limitation, cells, tissue, urine, sputum, serum or blood samples.

Anti-DXd antibodies can be used in any detection method, for example without limitation, Enzyme linked Immunosorbent Assay (ELISA), Radioimmunoassay (RAI), Chemiluminescent immunoassay (CLIA), immunohistochemistry (IHC) or fluorescence activated cell sorting (FACS). ELISA can be used to quantify the concentration of DXd in patient samples such as cells, tissue, urine, sputum, serum or blood samples. This detection method can involve the steps of contacting an ADC contained in a biological sample with an immobilized anti-DXd antibody, contacting the ADC-anti-DXd antibody complex with a detectably labeled antibody specific for the anti-DXd antibody and then detecting the label. The detection of the label can be qualitative or quantitative. Alternatively, the anti-DXd antibody could be directly labeled with a detectable label, therefore eliminating the method step of adding an additional or secondary antibody that is labeled.

ELISA can be used to quantify the concentration of DXd in cells, tissue, urine, sputum, serum or blood samples. After administration of an ADC to a subject—cells, tissue, urine, sputum, serum or blood samples can be obtained at various time points and the method steps of contacting the ADC or the drug released from the ADC with an anti-DXd antibody, and then detecting the anti-DXd antibody with a labeled secondary antibody and then quantifying the signal produced by the label.

Immunohistochemistry (IHC) can also be used when the biological sample is a cell or tissue sample. When the ADC is administered, it will specifically bind an antigen on a cell. In certain embodiments the cell can be a cancer cell. The cell can be obtained as part of a tissue sample, and the drug detected by contacting an anti-DXd antibody, then contacting the anti-DXd antibody with a secondary antibody that is labeled, and detection and/or quantification of the label.

### Pharmaceutical Compositions and Formulations

Also provided are compositions, including laboratory or pharmaceutical formulations, comprising an anti-DXd antibody or antigen-binding fragment thereof, or polynucleotides comprising sequences encoding an anti-DXd antibody or antigen-binding fragment. In certain embodiments, compositions comprise one or more antibodies or antigen-binding fragments that bind to DXd, or one or more polynucleotides comprising sequences encoding one or more antibodies or antigen-binding fragments that bind to DXd. These compositions can further comprise suitable carriers and acceptable excipients including buffers, which are well known in the art.

Pharmaceutical formulations of an anti-DXd antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. The formulations to be used are generally sterile. Sterility can be readily accomplished, e.g., by filtration through sterile filtration membranes.

### EXAMPLES

### Example 1. Immunizations with DXd Conjugated Antigens

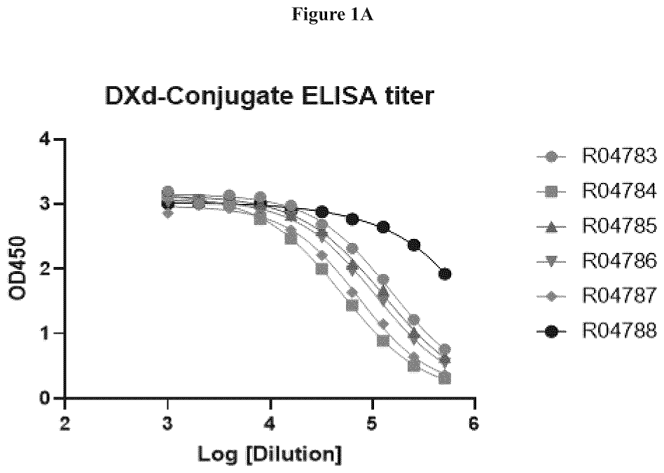

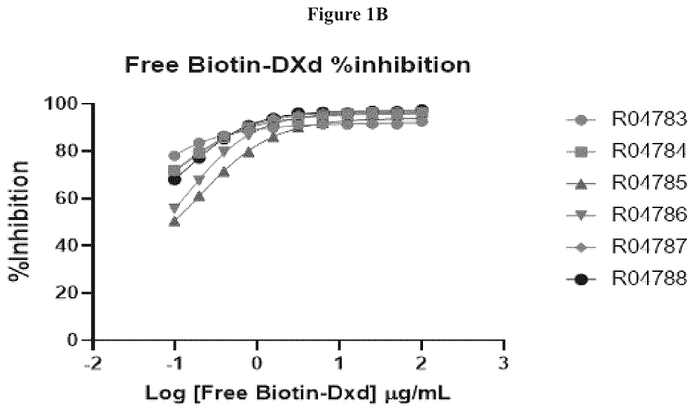

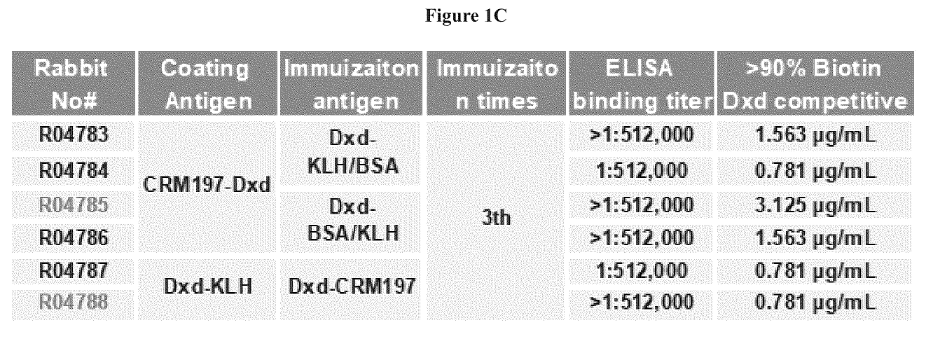

To generate Abs against DXd, cohorts of 6 New Zealand White (NZW) rabbits were immunized with DXd conjugated antigens with each cohort being subjected to an immunization strategy comprising a unique combination of DXd conjugated antigens, dose, injection route, adjuvant and immunization timing. A total of 2 or 4 animals in 2 cohorts were immunized. Animals received immunizations over varying periods between 0 and 56 days. To monitor immune responses, titrated serum was screened by ELISA as described below, typically after 21-56 days of 2-4 immunizations. Serum was screened for Ab binding to DXd conjugated antigens. DXd-specific Ab responses were measured in each animal, and animals with sufficient titers of anti-DXd Ig were selected for final boost 4 days before PBMC harvest to create B cell enrichment, screening and cloning. The immune titer profiles is shown in FIG. 1A-C.

### Example 2. Generation of B Cell Cloning and Screening of Antibodies to DXd

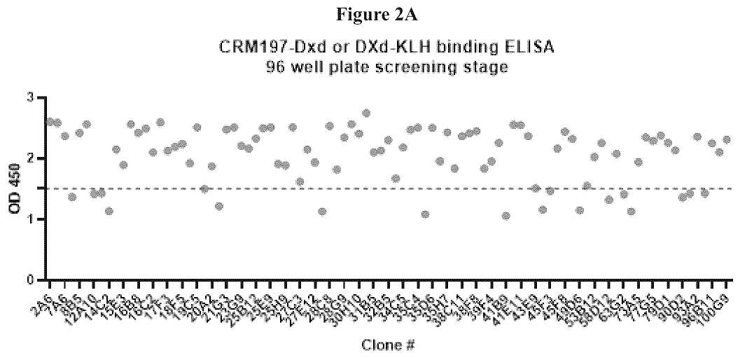

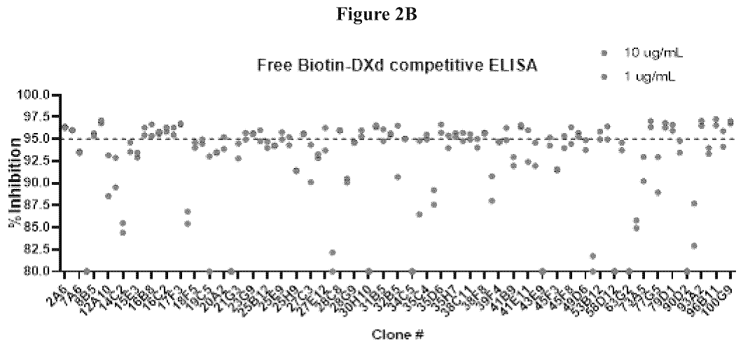

The B cells were isolated from the blood samples of rabbits using the rabbit B cell enrichment kit and antigen specific enrichment method. When the B cells were isolated, the B cells was seeded into a 96 well plate containing 5-10 B cells/well for culture with standard medium. The B-cell cultivation supernatants were removed for screening after 10-14 days culture. In general, the binding activity of the B-cell cultivation supernatants were assessed by the binding to the DXd conjugated antigens and free biotin-DXd competitive ELISA assay. A total of 75 positive clones met the screening criteria of the ELISA binding screening cut-off. OD450>1.5. A total of 65 positive clones met the screening criteria with free biotin-DXd competitive ELISA % inhibition cut-off: Inhibition >90% using 1 ug/ml free biotin-DXd as a competitive reagent as shown in FIGS. 2A-B.

### Example 3. Antibody VII and VL Gene Cloning and Sequencing

After the removal of the supernatant, the B-cell clones were lysed by 100 ml RLT buffer in 96-well round bottom plates. The mRNA containing lysates were subsequently transferred to 96-well deep well plates for mRNA isolation, cDNA synthesis and NGS sequencing. In general, total RNA of the cell lysates was prepared using the Total RNA Isolation Kit™ according to the manufacturer's instructions. The cDNA was generated by reverse transcription of the mRNA using the Super Script III first-strand synthesis SuperMix™ (Invitrogen) according to the manufacturer's instructions. The antibody sequences of representative antibodies are disclosed in Table 1.

### Example 4. EC50 Values for Binding of Recombinant Anti-DXd mAbs to DXd Conjugated Antigens

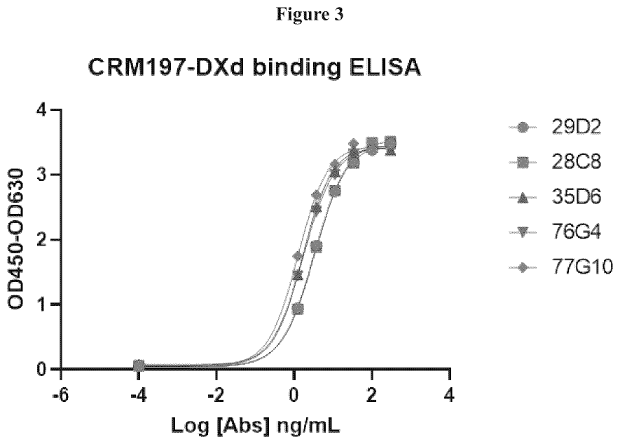

The antibody VH and VL genes were cloned into a rabbit IgG expression vector, and transfected into 293T cells for expression, after 1-14 days culture, the supernatant was harvested and run over a protein A affinity purification column. Several clones were selected for the recombinant anti-DXd mAbs expression and purification. After the purified mAbs were ready, the binding EC50 to DXd conjugated antigens was characterized using ELISA assay as shown in FIG. 3 and Table 3.

TABLE 3

| Clone # | EC50 (ng/mL) |
|---|---|
| 29D2 | 3.218 |
| 28C8 | 3.452 |
| 35D6 | 1.652 |
| 76G4 | 1.705 |
| 77G10 | 1.251 |

### Example 5. EC50 Values of Free Biotin-DXd Binding to Inhibit the Recombinant Anti-DXd mAbs to DXd Conjugated Antigens

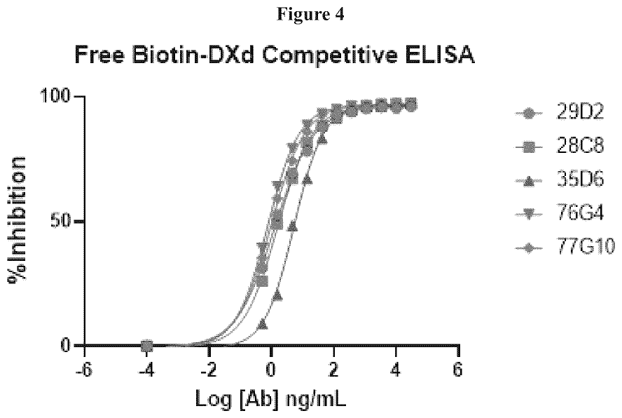

Briefly, DXd-conjugated antigen was coated onto high-binding 96-well plates (Corning) overnight at 4° C. Plates were blocked with BSA and washed, then 100 ul of the recombinant anti-DXd mAbs with different concentrations were added for 30-60 minutes on a plate shaker. In the meanwhile, 1 ug/ml free biotin-DXd was added with the recombinant anti-DXd mAbs. After washing, an appropriate streptavidin secondary antibody conjugated to horseradish peroxidase (HRP) was added, and the plates were developed with HRP substrate and the absorbance was measured. The % inhibition and the IC50 is shown in FIG. 4 and Table 4.

TABLE 4

| Antibodies | IC50 (ng/mL) |
|---|---|
| 29D2 | 1.263 |
| 28C8 | 1.576 |
| 35D6 | 5.245 |
| 76G4 | 0.753 |
| 77G10 | 0.953 |

### Example 6. Affinity Profiles of Recombinant Anti-DXd mAbs by SPR

The recombinant anti-DXd mAbs were generated in rabbit IgG format and characterized for the binding kinetics by SPR assays using BIAcore™ T-200 (GE Life Sciences). Briefly, Mouse Anti-Rabbit IgG Fc Antibody was immobilized on an activated CM5 biosensor chip (Cat. No. BR100530, GE Life Sciences). Purified rabbit antibodies were flowed over the chip surface and captured by an anti-rabbit IgG antibody. Then a serial dilution of KLH-DXd conjugated antigen was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of recombinant anti-DXd mAbs is shown below in Table 5.

TABLE 5

| | | ka | kd | KD | Rmax |
|---|---|---|---|---|---|
| Antibody | Antigen | (1/Ms) | (1/s) | (M) | (RU) |
| 28C8 | KLH-DXd | 1.09E+06 | 5.59E−08 | 5.14E−14 | 341.3 |
| 29D2 | KLH-DXd | 1.20E+06 | 1.31E−08 | 1.10E−14 | 220.9 |
| 35D6 | KLH-DXd | 9.30E+05 | 1.03E−08 | 1.11E−14 | 279.2 |
| 76G4 | KLH-DXd | 8.61E+05 | 7.54E−06 | 8.75E−12 | 85.8 |
| 77G10 | KLH-DXd | 3.28E+05 | 4.27E−07 | 1.30E−12 | 88 |
| 77G10 | KLH- | — | — | No Binding | — |
| | Control | | | | |
| | peptide | | | | |

1. An antibody or antigen-binding fragment thereof, comprising:
(i) a heavy chain variable region that comprises (a) a HCDR1 (Heavy Chain Complementarity Determining Region 1) of SEQ ID NO: 11, (b) a HCDR2 of SEQ ID NO: 12 and (c) a HCDR3 of SEQ ID NO: 13 and a light chain variable region that comprises: (d) a LCDR1 (Light Chain Complementarity Determining Region 1) of SEQ ID NO: 14, (e) a LCDR2 of SEQ ID NO: 15, and (f) a LCDR3 of SEQ ID NO: 16;
(ii) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 4, (e) a LCDR2 of SEQ ID NO: 5, and (f) a LCDR3 of SEQ ID NO: 6;
(iii) a heavy chain variable region that comprises (a) a HCDR1 of SEQ ID NO: 21, (b) a HCDR2 of SEQ ID NO: 22 and (c) a HCDR3 of SEQ ID NO: 23; and a light chain variable region that comprises: (d) a LCDR1 of SEQ ID NO: 24, (e) a LCDR2 of SEQ ID NO:25, and (f) a LCDR3 of SEQ ID NO: 26;
(iv) a heavy chain variable region that comprises (a) a HCDR1 of SEQ ID NO: 31, (b) a HCDR2 of SEQ ID NO: 32 and (c) a HCDR3 of SEQ ID NO: 33; and a light chain variable region that comprises: (d) a LCDR1 of SEQ ID NO: 34, (e) a LCDR2 of SEQ ID NO:35, and (f) a LCDR3 of SEQ ID NO: 36; or
(v) a heavy chain variable region that comprises (a) a HCDR1 of SEQ ID NO: 41, (b) a HCDR2 of SEQ ID NO: 42 and (c) a HCDR3 of SEQ ID NO: 43; and a light chain variable region that comprises: (d) a LCDR1 of SEQ ID NO: 44, (e) a LCDR2 of SEQ ID NO:45, and (f) a LCDR3 of SEQ ID NO: 46.

2. The antibody or antigen-binding fragment of claim 1, comprising:
(i) a heavy chain variable region (VH) comprising an amino acid sequence at least 90, 91, 92, 93, 94, 95, 96, 97, 98 or 99% identical to SEQ ID NO: 17, and a light chain variable region (VL) comprising an amino acid sequence at least 90, 91, 92, 93, 94, 95, 96, 97, 98 or 99% identical to SEQ ID NO: 18;
(ii) a heavy chain variable region (VH) comprising an amino acid sequence at least 90, 91, 92, 93, 94, 95, 96, 97, 98 or 99% identical to SEQ ID NO: 7, and a light chain variable region (VL) comprising an amino acid sequence at least 90, 91, 92, 93, 94, 95, 96, 97, 98 or 99% identical to SEQ ID NO: 8;
(iii) a heavy chain variable region (VII) comprising an amino acid sequence at least 90, 91, 92, 93, 94, 95, 96, 97, 98 or 99% identical to SEQ ID NO: 27, and a light chain variable region (VL) comprising an amino acid sequence at least 90, 91, 92, 93, 94, 95, 96, 97, 98 or 99% identical to SEQ ID NO: 28;
(iv) a heavy chain variable region (VH) comprising an amino acid sequence at least 90, 91, 92, 93, 94, 95, 96, 97, 98 or 99% identical to SEQ ID NO: 37, and a light chain variable region (VL) comprising an amino acid sequence at least 90, 91, 92, 93, 94, 95, 96, 97, 98 or 99% identical to SEQ ID NO: 38; or
(v) a heavy chain variable region (VH) comprising an amino acid sequence at least 90, 91, 92, 93, 94, 95, 96, 97, 98 or 99% identical to SEQ ID NO: 47, and a light chain variable region (VL) comprising an amino acid sequence at least 90, 91, 92, 93, 94, 95, 96, 97, 98 or 99% identical to SEQ ID NO: 48.

3. The antibody or antigen-binding fragment of claim 2, wherein one, two, three, four, five, six, seven, eight, nine, or ten amino acids within SEQ ID NO: 17, 18, 7, 8, 27, 28, 37, 38, 47 or 48 have been inserted, deleted or substituted.

4. The antibody or antigen-binding fragment of claim 1, that comprises:
(i) a heavy chain variable region (VH) that comprises SEQ ID NO: 17, and a light chain variable region (VL) that comprises SEQ ID NO: 18;
(ii) a heavy chain variable region (VH) that comprises SEQ ID NO: 7, and a light chain variable region (VL) that comprises SEQ ID NO: 8;
(iii) a heavy chain variable region (VH) that comprises SEQ ID NO: 27, and a light chain variable region (VL) that comprises SEQ ID NO: 28;
(iv) a heavy chain variable region (VH) that comprises SEQ ID NO: 37, and a light chain variable region (VL) that comprises SEQ ID NO: 38; or
(v) a heavy chain variable region (VH) that comprises SEQ ID NO: 47, and a light chain variable region (VL) that comprises SEQ ID NO: 48.

5. The antibody or antigen-binding fragment of claim 1, which is a monoclonal antibody, a chimeric antibody, a humanized antibody, a human engineered antibody, a single chain antibody (scFv), a Fab fragment, a Fab′ fragment, or a F(ab′)2 fragment.

6. The antibody or antigen-binding fragment of claim 1, wherein the antibody or antigen-binding fragment thereof has reduced glycosylation or no glycosylation or is hypofucosylated.

7. The antibody or antigen-binding fragment of claim 1, wherein the antibody or antigen-binding fragment thereof comprises increased bisecting GlcNac structures.

8. A diagnostic reagent comprising the antibody or antigen-binding fragment thereof of claim 1, further comprising a detectable label.

9. The diagnostic reagent of claim 8, wherein the detectable label is selected from the group consisting of a radiolabel, a fluorophore, a chromophore, an imaging agent, and a metal ion.

10. A method of detecting DXd in a biological sample comprising the steps of:
(a) administering an antibody drug conjugate containing DXd to a subject in need thereof;
(b) contacting a biological sample taken from the subject with the anti-DXd antibody of claim 1; and
(c) detecting a label.

11. The method of claim 10, wherein the biological sample is a tissue, a cell or a fluid.

12. The method of claim 11, wherein the fluid is serum, plasma or whole blood.

13. The method of claim 10, wherein the anti-Dxd antibody is directly labeled.

14. The method of claim 10, wherein a labeled secondary antibody contacts the anti-DXd antibody.

15. An isolated nucleic acid that encodes the antibody or antigen-binding fragment of claim 1.

16. A vector comprising the nucleic acid of claim 15.

17. A host cell comprising the nucleic acid of claim 15.

18. A process for producing an antibody or antigen-binding fragment thereof comprising cultivating the host cell of claim 17 and recovering the antibody or antigen-binding fragment from the culture.

19. A host cell comprising the vector of claim 16.

20. A process for producing an antibody or antigen-binding fragment thereof comprising cultivating the host cell of claim 19 and recovering the antibody or antigen-binding fragment from the culture.
